(19) United States
(12) Patent Application Publication
(10) Pub. No.: US 2026/0155240 A1
(43) Pub. Date: Jun. 4, 2026

(54) Vessel Morphology Radiomic Phenotypes

(71) Applicant: Picture Health Inc., Cleveland, OH (US)

(72) Inventors: Nathaniel M. Braman, Cleveland, OH (US); Kai Zhang, Santa Clara, CA (US); Anant Madabhushi, Atlanta, GA (US)

(21) Appl. No.: 19/406,609

(22) Filed: Dec. 2, 2025

Related U.S. Application Data
(60) Provisional application No. 63/727,570, filed on Dec. 3, 2024.
(60) Provisional application No. 63/782,513, filed on Apr. 2, 2025.
(60) Provisional application No. 63/796,995, filed on Apr. 29, 2025.
(60) Provisional application No. 63/804,866, filed on May 13, 2025.
(60) Provisional application No. 63/870,954, filed on Aug. 27, 2025.

(51) Int. Cl. G16H 30/40; G06N 20/00; G16H 15/00; G16H 20/00; G16H 50/20; G16H 50/70

(52) U.S. Cl. CPC G16H 30/40; G06N 20/00; G16H 15/00; G16H 20/00; G16H 50/20; G16H 50/70

(57) ABSTRACT

Biomarkers for treatment response and other outcomes in the treatment of lesions, such as cancerous tumors, are derived from routine clinical medical images, such as computed tomography (CT) and magnetic resonance imaging (MRI) scans, using radiomic machine learning techniques. The biomarkers are based, at least in part, on phenotypes derived from vessel morphology features descriptive of vasculature associated with a lesion shown in the medical images and may also include other radiomic and pathomic types of features. The biomarkers and phenotypes may be used at multiple points in the treatment process, and treatment decisions may be based on changes in radiomic features or phenotypes longitudinally through the course of treatment. The biomarkers and phenotypes may also be used in clinical trials, to establish trial parameters, patient enrollment, drug dosage, and other trial parameters.

### CROSS-REFERENCE TO RELATED APPLICATIONS

This application claims priority to, and the benefit of, U.S. Provisional Application No. 63/870,954, filed Aug. 27, 2025; U.S. Provisional Application No. 63/804,866, filed May 13, 2025; U.S. Provisional Application No. 63/796,995, filed Apr. 29, 2025; U.S. Provisional Application No. 63/782,513, filed Apr. 2, 2025; and U.S. Provisional Application No. 63/727,570, filed Dec. 3, 2024. All of those applications are incorporated by reference herein in their entireties.

### TECHNICAL FIELD

Embodiments of the invention relate to establishing biomarkers for cancer severity, treatment response, and prognosis and to systems and methods that use these biomarkers. The biomarkers are derived from image-based radiomic vascular morphology features.

### BACKGROUND

Traditionally, medical images, such as X-rays, CT scans, MRI scans, stained micrographs of tissue, and the like are interpreted qualitatively by radiologists, pathologists, and other medical providers. Such qualitative analyses may, for example, appreciate a bone fracture, the presence and certain qualities of a lung or breast tumor, or, by microscopic examination, the nature of the cells in a tissue sample. More recently, physicians, scientists, and engineers have understood such images as containing quantitative data that can be extracted from the images and processed using machine learning (i.e., artificial intelligence) techniques. When this is done, large numbers of quantitative features are extracted from the image and used with a machine learning model to make some sort of prediction or judgment about the image or about the patient of whom the image was taken. These types of techniques are often referred to as “radiomics” when the source medical images arise from radiology, and “pathomics” when the source medical images arise from pathology. In both cases, the -omics suffix, originally borrowed from the term “genomics,” denotes the often-vast scale of the data extracted and used in the processes.

U.S. Pat. No. 10,846,367 discloses a type of radiomic features that describe the morphology of the blood vessels associated with a lesion. These radiomic vessel morphology features, which the '367 patent refers to as quantitative vessel tortuosity (QVT) features, are extracted in great quantities from radiological medical images, such as computed tomography (CT) and magnetic resonance imaging (MRI) scans and are used by machine learning models to make predictions of various sorts. For example, the '367 patent uses QVT to predict recurrence in non-small-cell lung cancer (NSCLC).

In simple terms, vessel morphology features describe the physical and organizational characteristics of the vasculature. For example, such features may describe how “twisted” the vessels are, the number of branches in the vasculature, etc. Machine learning applications may use hundreds of different features related to vessel morphology, taken at many points along the vasculature, to make a prediction, either alone or in combination with other features.

While the concept of radiomic vessel morphology may be simple, its execution is complex: the radiomic features extracted and used in radiomic processes are often not perceptible to the human eye, and the radiomic features are typically extracted in such great numbers that the processes cannot be performed without a machine. Moreover, supervised machine learning is most frequently used in radiomic and pathomic work: the machine learning models that are used typically must be specially trained to make whatever predictions are desired, which requires a careful process using data from patients with known outcomes relative to the desired predictions.

Vessel morphology features can often be tied to biological hypotheses that explain the effects of vessel morphology on disease. For example, previous work has shown that tumors with tortuous, disorganized vasculature are less likely to respond to some forms of treatment. There are many hypotheses as to why this is true. For example, the tortuous, disorganized vasculature may make it less likely that therapeutic agents will actually reach the tumor. Additionally or alternatively, the chaotic vasculature may result in less consistent perfusion, which may result in a hypoxic tumor microenvironment. That hypoxic tumor microenvironment may lessen the effectiveness of treatment by several different mechanisms. Hypotheses like these give vessel morphology features particular advantages when used in machine learning applications, because determinations and predictions made by a machine learning model based on such features may be more easily interpreted and explained, which can increase physician and patient confidence in those determinations and predictions. By contrast, results reached using other image-based features in machine learning applications, such as image texture features, may not be so easily explainable.

In the treatment of cancerous tumors, clinicians and patients face myriad decisions. For example, depending on the type and stage of a tumor, radiation, chemotherapy, and immunotherapy may all be treatment options. However, relatively few biomarkers or assays exist to determine which patients are more likely to respond to which treatments. Moreover, the decision to provide a patient with one type of treatment may foreclose the possibility of using other treatments. There are pragmatic concerns as well: some treatments, like immunotherapies, are expensive and have relatively poor response rates, making it especially helpful to select patients most likely to benefit from the treatment.

Recent clinical trial results with ivonescimab, a bisensitive synthetic antibody that targets both vascular endothelial growth factor (VEGF) and programmed cell death protein 1 (PD-1), reemphasize the importance of tumor-associated vasculature in cancer treatment, both alone and in combination with other targets. However, as with many other forms of cancer treatment, there are few identified biomarkers or assays to tell clinicians which patients are most likely to respond to anti-angiogenic and combined treatments.

### BRIEF SUMMARY

In general, aspects of the invention relate to methods for creating and using biomarkers based on sets of radiomic vessel morphology features. These biomarkers are typically created by extracting sets of vessel morphology features from three-dimensional vascular segmentations derived from medical images of patients. Typically, the medical images show a lesion or tumor, and the vasculature is associated with that lesion or tumor. The extracted sets of vessel morphology features are processed using machine learning, often unsupervised machine learning, such as clustering, to identify patient groups based on values of the extracted vessel morphology features. Although it may not be necessary to know patient outcomes in order to identify the patient groups, typically, the extracted vessel morphology features have shown an association with particular outcomes or outcome metrics. Optionally, dimensionality reduction and other such techniques may be used to reduce the extracted set of vessel morphology features to a subset. The resulting phenotypical set of vessel morphology features can then be used on medical images and vascular segmentations of patients with unknown outcomes, with the values of the extracted features indicating potential outcomes and risks and acting as an aid in clinical and research decision making. The resulting phenotypes may be used at various stages of treatment, including to determine whether a patient is likely to respond to a particular treatment; to select a treatment among various treatment options, both in patients who are naïve to treatment and in patients who have already had one or more courses of treatment; to monitor the progress of treatment; to select patients for clinical trials and other research and investigatory processes; and to evaluate the safety, the efficacy or the mechanism of action of a treatment.

In many cases, the values of the extracted set of radiomic vessel morphology features may be reduced to a vessel morphology score or other, similar form of summary metric that indicates which outcome-group the patient is most likely to fit within. A vessel morphology score may be reported, much in the same way as a traditional laboratory test, against “reference ranges” for such scores that define the bounds of the various outcome-groups.

Vessel morphology features may be divided into various types or categories, including, e.g., branching features, torsion features, curvature features, radius features, vessel volume features, inflection point features, features derived from Frenet-Serret frame vectors, and bending energy features. In some cases, a “composite” set of vessel morphology features that includes multiple categories of features may be used. In other cases, a “component” set of vessel morphology features that includes only a single category or the use of a group of sets, each set including only a single category, may be used.

As one particular example, a method according to one aspect of the invention may involve, using machine learning, grouping first medical images into two or more groups on the basis of a set of radiomic vessel morphology features extracted from the first medical images or from a vascular segmentation derived from the first medical images. On the basis of the grouping, a phenotypical set of radiomic vessel morphology features is defined. The phenotypical set of radiomic vessel morphology features is (1) at least a subset of the set of radiomic vessel morphology features extracted from the first medical images, and (2) medically predictive of the two or more groups.

Given the above method, a method for using the resulting phenotypical set of radiomic vessel morphology features may involve extracting the phenotypical set of radiomic vessel morphology features from a second medical image or images of a patient, or from a vascular segmentation derived from the medical image or images. Based on values of the extracted phenotypical set of radiomic vessel morphology features, a medical prediction concerning the patient is then made.

Once such a biomarker is established, that is, once a phenotypical set of radiomic vessel morphology features is established, it may be used in a broad array of contexts, typically without having to train a supervised machine learning model for a particular disease or type of cancer. While specific biomarkers of this type may be established for different types of treatments, different stages of treatment, and specific patient characteristics, in many cases, particularly if a biomarker is established based on broad data, it may be possible to use the same biomarker, i.e., the same phenotypical set of radiomic vessel morphology features, at different points in treatment and in different contexts. For example, the same biomarker may be used longitudinally through an entire course of treatment.

As another example of a method for using such a biomarker according to an aspect of the invention, a method may comprise extracting a set of phenotypical radiomic vessel morphology features. The set of phenotypical radiomic vessel morphology features may be extracted from (1) a three-dimensional segmentation of vasculature, (2) a portion of the three-dimensional segmentation of the vasculature, or (3) a transform or projection of the three-dimensional segmentation of the vasculature. The three-dimensional segmentation of the vasculature is constructed from one or more medical images of a patient. Based on the values of features within the extracted phenotypical set of radiomic vessel morphology features, the method may be used in evaluating the safety, efficacy, or mechanism of action of a medical treatment in the patient.

This method may also comprise computing a vessel morphology score calculated based on values of the extracted radiomic features and using that vessel morphology score in the evaluation. That score may be reported against reference ranges. In some cases, the vessel morphology score may be a “composite” score that uses the values of different types or categories of radiomic features, while in other cases, it may be a “component” score that uses only one type or category of radiomic feature. In some cases, a panel or collection of component scores may be used.

In some embodiments, the medical treatment may be a drug treatment. In other embodiments, the medical treatment may be, e.g., a surgical or radiological intervention.

Other aspects of the invention relate to apparatus and systems for carrying out the kinds of methods described above, as well as to machine-readable media with sets machine-readable instructions thereon that, when executed, cause a processor to perform the methods.

Other aspects, features, and advantages of the invention will be set forth in the following description.

### BRIEF DESCRIPTION OF THE DRAWING FIGURES

The invention will be described with respect to the following drawing figures, in which like numerals represent like features throughout the description, and in which:

FIG. 1 is a flow diagram of a method for establishing biomarkers and assays using vessel morphology features according to one embodiment of the invention;

FIG. 2 is a flow diagram of a method for performing an assay for the biomarkers of FIG. 1;

FIG. 3 is a schematic diagram of a system according to one embodiment of the invention;

FIG. 4 is a flow diagram of a method for using tumor phenotypes and change in phenotype during a treatment process;

FIG. 5 is an illustration of a vessel morphology phenotype report;

FIG. 6 is an illustration of a vessel morphology phenotype report that includes measures of phenotypical change;

FIG. 7 is a flow diagram of a method for creating biomarkers based on individual types of vessel morphology features;

FIG. 8 is an illustration of a graphical user interface that displays data on vessel morphology in conjunction with other information;

FIG. 9 is an illustration of a portion of the graphical user interface of FIG. 8 that displays a segmented tumor in three dimensions, showing its location within the lungs;

FIG. 10 is an illustration of a portion of the graphical user interface of FIG. 8 that displays a segmented tumor and its vasculature in three dimensions, illustrating one vessel morphology component score, torsion, using color and/or intensity gradients along the vasculature at a first point in time;

FIG. 11 is an illustration similar to that of FIG. 10 illustrating the same vessel morphology component score, torsion, at a second point in time;

FIG. 12 is an illustration similar to that of FIGS. 10 and 11, illustrating the same vessel morphology component score, torsion, at a third point in time; and

FIGS. 13A and 13B are three-dimensional renderings of segmentations of two different vessel morphologies associated with tumors, one (FIG. 13A) with which the patient had an overall survival greater than 22 months, and another (FIG. 13B) with which the patient died within two months.

### DETAILED DESCRIPTION

### Concepts and Definitions

This description relates to biomarkers and assays that rely, at least in part, on vessel morphology features extracted from medical images. A “biomarker” or “signature,” as those terms are used here, refer to a sign of a normal or abnormal biological process, or of a condition or disease. An “assay” is a test for a particular process or condition. For example, an assay may test for the presence of a particular biomarker.

The terms “drug,” “therapeutic agent,” and “agent” are used interchangeably to refer to any substance that causes an improvement in a clinically recognized characteristic or symptom associated with a disease or condition, or that is being investigated to determine whether it may cause such an improvement. The drug or therapeutic agent may be a natural product, a biologic, a chemical compound, or any other substance.

“Features,” in this description, are pieces of quantitative information extracted or computed from medical images or datasets derived from medical images. The term “features” should be construed to include statistics and other aggregate descriptors of features. Features may be sub-visual or not perceptible to the human eye, and this description will assume that features cannot be extracted using mental processes or pencil and paper.

“Medical images,” as the term is used here, are images of the body or portions of it created for clinical or research medical purposes, including images of the interior of the body and its tissues, as well as documentary photographs and other types of images taken for clinical medical or research purposes. Medical images may be divided into several relevant types, and unless further qualified, the term should be construed to encompass all types. Radiology images are medical images derived from radiology studies of the body or parts of it, including x-rays, CT scans, MRI scans, PET scans, and variations or applications of those modalities, like dynamic contrast enhanced MRI (DCE-MRI), mammography, and breast tomosynthesis. Pathology images are medical images derived from studies of the cells and tissues of the body, often at greater-than-visual magnification. Pathology images include, but are not limited to, documentary photographs of the body and its organs and tissues, whole-slide images (WSIs) of tissues, as well as images of tissue microarrays. WSIs may be stained to accentuate a particular set of cellular or tissue features, e.g., with hematoxylin and eosin (H&E) stain or other stains.

As was noted above, historically, medical images have been interpreted qualitatively by radiologists, pathologists, and other physicians. Portions of this description are based on the premise that medical images contain quantitative data that can be extracted and used by machine learning models. “Radiomics” is the branch of medical machine vision and machine learning in which features are extracted from radiology images and used with machine learning models to make medical predictions. “Pathomics” is the branch of medical machine vision and machine learning in which features are extracted from pathology images and used with machine learning models to make medical predictions. For this reason, the term “features” may be qualified with “radiomic” or “pathomic” to describe the origin of the features in question. In both cases, the “-omics” suffix, borrowed from the term “genomics,” is used to indicate the large scale at which features are extracted and used. Radiomics and pathomics cannot be done in the mind or with pencil and paper for many reasons; for example, many of the features in question are sub-visual and/or not perceivable by the human eye. The scale at which features are extracted-sometimes hundreds or thousands of features from hundreds or thousands of locations within a single image—also makes it impractical to perform radiomic and pathomic methods without a machine or machines.

“Vessel morphology features,” as the term is used here, refers to features that describe the form of blood vessels and the organization of groups of blood vessels. Vessel morphology features may be considered to be a subset of radiomic features, as they are typically extracted from radiology images. These features may, for example, describe the tortuosity (i.e., twistedness), curvature, volume, and branching of the blood vessels. “Blood vessels,” as the term is used here, encompasses all forms of blood vessel, including arteries, veins, arterioles, venules, capillaries, etc., although the actual blood vessels that are used in any particular embodiment or application may depend on the resolution and other capabilities of the imaging modality and other factors. Not all types of blood vessels are necessarily visible (i.e., perceptible) using all imaging modalities. The blood vessels will typically be associated with a lesion or tumor, and may, in some cases, be referred to as lesion-associated vasculature or tumor-associated vasculature, as the case may be. However, there is no particular limitation as to how far from a lesion or tumor the vasculature need be, or how directly or indirectly that vasculature is connected to or associated with a lesion or tumor. In some cases, the vasculature in question may not have a direct association with a lesion or tumor, or, at least, not an association that can be readily discerned by a human observer. In this description, “vessel morphology features” is intended to refer to a genus-subset of radiomic features, of which the kind of quantitative vessel tortuosity (QVT) features described above are a subset or species.

Embodiments of the invention may use other types of features, and the term “feature” should be read broadly enough to cover other types of features. For example, as will be described below in more detail, vessel morphology features may be used with other types of radiomic features, such as texture features.

“Extraction” and “feature extraction” as those terms are used here, refer to processes and techniques that either define feature values by taking quantitative information from a medical image or derive feature values by calculation using quantitative information from a medical image. Deriving feature values by calculation includes processes and techniques for calculating statistics and other aggregate descriptors of features.

Embodiments of the invention use machine models to establish biomarkers and assays, to conduct assays for particular biomarkers, and to make predictions based on the biomarkers and assays, among other functions. “Machine model” and “machine learning model” are used interchangeably in this description to refer to a computer program or algorithm that has been trained to make a prediction or predictions based on various types of input data. Machine learning models may be of various types, and unless the type is specified, the term should be considered to be generic. For example, a “deep learning model” is a machine model that uses an artificial neural network, such as a convolutional neural network (CNN) or a transformer, to make its predictions. However, unless the term “model” is qualified in such a way as to indicate its nature (e.g., “a machine learning model”), the term should be interpreted more broadly. For example, a nomogram is a type of non-machine model that may be used in and with embodiments of the invention.

The term “prediction” is used in this description to describe most forms of output from the kinds of machine models described here. This is because the output of all machine models is, at some level, uncertain and predictive in nature. For example, a machine model may be essentially guessing the correct output to any input based on similar training data. A “longitudinal prediction” is a prediction that concerns the evolution or progression of a patient or group of patients over time. A longitudinal prediction may or may not use longitudinal data. For example, a longitudinal prediction may use patient data from one or several points in time to predict the evolution of a patient's disease over time.

The term “lesion,” as used here, refers to any kind of damage to, or disease in, tissue. A lesion may be benign, or it may be malignant (i.e., cancerous). Thus, the term “lesion” should be thought of as generic. By contrast, a “tumor” or “cancerous tumor” is a malignant form of lesion. In general, the methods and systems described here are applicable to any type of solid tumor, including solid tumors of the lung, breast, ovaries, colon, oropharynx, and pancreas.

### Establishing Biomarkers and Assays

Recent advances in the field of radiomics have demonstrated the ability of quantitative information from standard radiologic images to aid in therapeutic decision support. Traditionally, these approaches involve the extraction of a number of subvisual measurements, e.g., image texture, from one or more lesions within a radiology scan, followed by the training of a machine learning model to predict the outcome of a particular treatment. A similar, more recent strategy is the training of a deep learning model like an artificial neural network to learn its own image features and how they can be applied to a medical prediction task. A commonality of both of these strategies is their training is achieved through supervised learning, a machine learning strategy where a model is presented with input image-derived data for a group and the true outcomes. During this process, the model is trained to use the input data to predict the correct outcome labels as accurately as possible. Training a predictive radiomics model in this fashion requires both training and evaluation data, i.e., data from patients who received the specific treatment for which a prediction is desired. An advantage of supervised radiomics models is that they can be finely tuned to a particular therapy and thus provide decision support specific to the therapy on which they were trained. However, a consequence of this specificity is that a new model must be developed for every alternative treatment option, each of which requires a new patient dataset assembled from recipients of that therapy. This is especially constraining in the case of emerging therapies and in the clinical trial setting, where the pool of available data from patients who have received that treatment is inherently scarce.

Using biomarkers and assays to classify a patient or a lesion—sometimes referred to as “phenotyping”—is an alternative strategy to providing therapeutic decision support. Phenotyping is a hypothesis-driven process to discover or measure an inherent biological categorization relevant to tumor response and outcomes. In general, phenotypes do not require a dataset of patients who received a particular treatment or patients with known outcomes for development, nor are their applications inherently constrained to a particular treatment regimen. Phenotypes can be tested broadly and may be effective in any context where their underlying biologic basis is relevant to a therapy's mechanism of response-making them an asset within the drug development setting as well as in clinical settings.

As one example, conventional phenotypes, developed over decades of research, categorize a tumor and its microenvironment into either an immune-inflamed, an immune-excluded, or an immune-desert phenotype. These phenotypes have accordingly been shown to provide value in a variety of therapeutic agents that operate on the body's immune mechanisms. However, conventional phenotypes do not use radiomic features, such as vessel tortuosity features, and while there have been attempts to construct radiomic biomarkers and phenotypes, these attempts rely on types of features, like image texture features, that are not deeply grounded in interpretable biological and physiological truths.

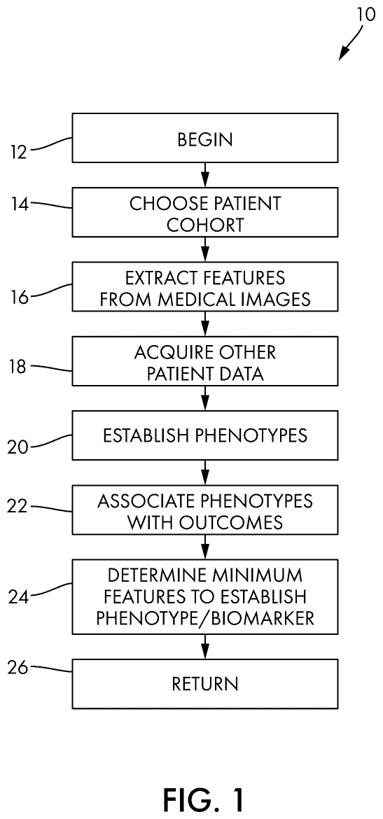

FIG. 1 is a schematic flow diagram of a method, generally indicated at 10, for establishing biomarkers and assays that are based, at least in part, on vessel morphology features. As those of skill in the art will note, there are multiple ways to combine biological measurements to establish a phenotype. Thus, method 10 is only one example of a method that may be used to establish biomarkers, assays, and related phenotypes. Method 10 begins at 12 and continues with task 14.

In task 14, a cohort of patients is selected. In some embodiments, the cohort of patients is a retrospective cohort with relevant diagnoses, available medical imaging and other pertinent medical history data and, preferably, known responses to treatments and known outcomes. For example, if method 10 is used to establish a biomarker for treatment response in NSCLC, one would select a cohort of patients with NSCLC, in some cases diagnosed at a particular stage, and with known outcome metrics, such as known overall survival (OS) and progression-free survival (PFS). The selected patient cohort preferably has an adequate number of medical images, such as CT or MRI scans, in the case of NSCLC, taken at appropriate points in treatment. Once the patient cohort is selected, relevant medical images are also defined. For example, a cohort of patients with advanced NSCLC could be selected, and CT scans prior to the administration of immune checkpoint inhibitor (ICI) monotherapy could be used as medical images for purposes of method 10.

However, as noted above, biomarker- and phenotype-based approaches may not require such specific information to be known for the selected cohort. For example, it may not be necessary for all of the cohort to have undergone a specific type of treatment, and it may not be necessary for outcome data to be known. However, the retrospective cohort of patients selected in task 14 would be generally similar to a cohort of patients in which any eventual biomarker is actually used. Once a cohort of patients is selected, the data from those patients is used for the remainder of the tasks of method 10.

Method 10 continues with task 16. In task 16, features are extracted from medical images. Although task 16 is described here as a part of method 10, in some embodiments, it may be done in a way that is disconnected from, or out-of-sequence with, the other tasks of method 10. That is, the feature extraction task itself may be performed at a different time than other tasks of method 10, on different equipment than other tasks of method 10, or by a different entity. For example, a feature extraction apparatus or software routine may be included in a medical imaging scanner, such as a CT or MRI scanner. Alternatively, a separate computer system or software module may be programmed to automatically extract a predetermined set of features from every medical image or scan placed in a radiology information system (RIS) or other such medical image repository, or, at least, from every medical image or scan meeting certain predefined criteria.

If task 16 is done automatically or in some other way, method 10 would begin by accessing the pre-extracted feature sets. If feature extraction is performed as a defined part of method 10, it may be done in any way known in the art.

More specifically, in task 16, at least a set of vessel morphology features is extracted. Depending on the type of features that are to be extracted, task 16 may operate on a single medical image, on a collection of medical images, or on a pre-prepared segmentation of structures shown in medical images. Thus, in some cases, method 10 may include additional tasks prior to task 16 to prepare for feature extraction.

For example, the medical images that are used as input may require quality control or standardization processes before feature extraction can take place. For example, images may need to be upsampled or downsampled to a standard resolution, cropped, or to have artifacts removed. As another example, pathology images may be subjected to a quality control process or application prior to use, like the application disclosed in U.S. Pat. No. 10,861,156, the contents of which are incorporated by reference in their entirety.

The medical images may be in essentially any usable format, including Digital Communications in Medicine (DICOM) format, NIFTI format, TIFF format, SVS format, JPG format, etc., and may be associated with metadata describing the nature of the study that produced the image, patient information, etc. In non-clinical uses, it may be necessary or desirable to anonymize data so as to protect patient identity.

In some cases, the “preprocessing” steps performed on a medical image may be a function of the file format in which the medical image is stored. For example, in DICOM whole slide imaging of pathology images, lower-resolution versions of a whole slide image are automatically computed and stored in a “pyramid” of image data that facilitates retrieval of image data at arbitrary resolutions. Thus, when using DICOM-format images, e.g., it may not be necessary to perform upsampling or downsampling.

As was noted briefly above, another usual predicate to feature extraction is segmentation. “Segmentation” is a general term referring to the process of distinguishing the structures in a medical image from one another. In this case, the vasculature, and in many cases, all of the structures in a medical image or collection of medical images, including cancerous lesions, are segmented. This description will generally assume that segmentation is performed automatically, although in some embodiments, a user could be prompted to manually segment an image using, e.g., a graphical user interface (GUI). A user could also be prompted to provide annotations that the system then uses to create a segmentation.

Automatic segmentation methods frequently use deep-learning machine models, such as those based on CNNs, to create a segmentation. Deep-learning machine models specialized for image segmentation, like U-nets, may be used, as may transformers and other types of deep-learning machine models. Other types of segmentation models and approaches that do not rely on deep learning machine models could also be used, including thresholding, active contour, and region-growing approaches. In some embodiments, the machine learning model that produces the prediction or predictions may be able to process the medical image and extract the features without a separate segmentation step. This is particularly true when the machine learning model used to produce the prediction or predictions is a deep learning model.

Additional steps may also be taken, like the use of a fast-march algorithm to identify the centerlines of the vessels, and steps to connect disconnected vessel portions. In some cases, it may be helpful to implement a smoothing algorithm.

When extracting vessel morphology features, a three-dimensional segmentation of the vasculature, or a portion of such a segmentation, is typically used. The segmentation is typically also further processed to identify vessel centerlines and to connect disconnected vessel portions, as described above. Depending on the particular features that are to be extracted, a two-dimensional segmentation or a slice of the three-dimensional segmentation may also be used. Additionally, projections and transforms of a three-dimensional vessel segmentation may be used for the extraction of some types of features. For example, some features may be extracted from a projection of the three-dimensional segmentation onto a two-dimensional plane (e.g., an XY plane projection, an XZ plane projection, a YZ plane projection, etc.). In some cases, features may be extracted from a transform of the three-dimensional segmentation or a transform of a slice or plane projection of the three-dimensional segmentation. See, e.g., Braman, N. et al., “Vascular Network Organization via Hough Transform (VaNgOGH): A Novel Radiomic Biomarker for Diagnosis and Treatment Response” in Medical Image Computing and Computer Assisted Intervention—MICCAI 2018 (eds. Frangi, A. F., et al.), pp. 803-811 (Springer, 2018).

Types of vessel morphology features that may be extracted include vessel curvature (e.g., defined using the radius of a circle fit to several points along the centerline of a vessel; see U.S. Pat. No. 9,595,103); vessel tortuosity (e.g., defined based on the length along the centerline of a vessel between two points compared to the distance between those two points; see U.S. Pat. No. 9,595,103); the radii of curved segments of vasculature across a branch; vascularization (e.g., defined as the number of vessels entering the tumor); and overall vessel volume. Additional vessel morphology features that may be extracted include the number of inflection points in a vessel; the number of vessel branches; and branch lengths. All features may be extracted at the global level, at the branch level, or at any other scale or level that is convenient or helpful. For each feature, statistics like the kurtosis, maximum, mean, median, skew, standard deviation, and quantiles (e.g., 5^{th} percentile, 25^{th} percentile, 50^{th} percentile, and 75^{th} percentile) may be extracted as separate features. For example, all of the above statistics may be extracted globally and for each vascular branch, followed by a second statistical summarization metric. That is, once features have been established for a single branch or region of vasculature, it may be necessary or desirable to summarize those features in a single value using a statistic. One example of this would be a calculation of the mean curvature of each branch, followed by the calculation of the standard deviation of these curvatures across all branches of the vessel network.

In addition to vessel morphology features, other radiomic and pathomic features may be extracted from medical images in task 16 and used in method 10 to establish a biomarker, assay, or phenotype. Examples of other radiomic features that may be used include histogram features, textural features, filter- and transform-based features, and size- and shape-based features. (Some vessel morphology features can be considered to be a special case of size- and shape-based features.) The classification of various radiomic features may vary depending on the authority one consults; the categories used here should not be considered a limitation on the range of features that could potentially be used. As noted above, the term “feature” should also be construed to include statistics that describe or summarize extracted features. For example, it may be convenient to use the mean, maximum, minimum, variance, skewness, kurtosis, etc. of a particular feature, globally or in a particular neighborhood or portion of the medical image, as input to a model. “Raw” features extracted from a medical image may also be normalized or otherwise manipulated before further use.

Histogram features use the global or local gray-level histogram, and include gray-level mean, maximum, minimum, variance, skewness, kurtosis, etc. Measures of energy and entropy may also be taken as histogram or first-order statistical features. Texture features explore the relationship between voxels and include the gray-level cooccurrence matrix (GLCM), the gray-level run-length matrix (GLRLM), gray-level size zone matrix (GLSZM), and gray-level distance zone matrix (GLDZM). Co-occurrence of local anisotropic gradient orientations (COLLAGE) features are another form of texture feature that may be used. (See Prasanna, P. et al., “Co-occurrence of local anisotropic gradient orientations (collage): distinguishing tumor confounders and molecular subtypes on MRI,” in Medical Image Computing and Computer-Assisted Intervention—MICCAI 2014 (eds. Golland, P. et al.), pp. 73-80 (Springer, 2014).) Filter- and transform-based features include Gabor features, a form of wavelet transform, and Laws features.

Pathomic features may include, e.g., features of global and local graphs of the locations of nuclei, nuclear shape features, nuclear orientation entropy, and nuclear texture. Pathomic features may also include measures of other types of cells and structures, including, e.g., graphs and measures of tumor-infiltrating lymphocytes or measures of collagen fiber orientation, as well as statistics and graphs descriptive of these. Pathomic features may also include nuclear shape features, such as nuclear perimeter, minimum and maximum radii, smoothness, and Fourier transform of the nuclear contour (see, e.g., Lu, C. et al. “Nuclear shape and orientation features from H&E images predict survival in early-stage estrogen receptor-positive breast cancers,” Laboratory Investigation 98, pp. 1438-1448 June, 2018); nuclear texture features, such as gray-level co-occurrence features (Ibid.); global graphs of nuclei (see, e.g., Wang, X. et al. “Prediction of recurrence in early stage non-small cell lung cancer using computer extracted nuclear features from digital H&E images,” Scientific Reports 7:13543, October 2017); cell cluster graphs (i.e., local graphs, see, e.g., Ali, S. et al., “Cell cluster graph for prediction of biochemical recurrence in prostate cancer patients from tissue microarrays,” Proc. SPIE 8676, Medical Imaging 2013 March, 2013); cell orientation entropy (CORE; see, e.g., Lee, G. et al., “Cell Orientation Entropy (COrE): Predicting Biochemical Recurrence from Prostate Cancer Tissue Microarrays,” in Medical Image Computing and Computer Assisted Intervention—MICCAI 2013 (eds. Mori, K., et al.), pp. 396-403 (Springer, 2013)); local co-occurrence of cell morphology (LOCOM; see, e.g., Lu, C. et al., “A prognostic model for overall survival of patients with early-stage non-small cell lung cancer: a multicentre, retrospective study,” Lancet Digit. Health 2, e594-606, November 2020); feature-driven local cell clusters (FLocK; see Lu, C. et al., “Feature-driven local cell graph (FLocK): New computational pathology-based descriptors for prognosis of lung cancer and HPV status of oropharyngeal cancers,” Med. Image Analysis 68, November 2020); peri-nuclear pathomics (PNP; see, e.g., Wang, X. et al., “A prognostic and predictive computational pathology image signature for added benefit of adjuvant chemotherapy in early stage non-small-cell lung cancer,” eBioMedicine 69, July 2021); cell run length, which quantifies connectivity and branching patterns of cellular graphs; multinucleation index (MuNI; see, e.g., Koyuncu, C. et al., “Computerized tumor multinucleation index (MuNI) is prognostic in p16+ oropharyngeal carcinoma,” J. Clinical Investigation 131 (8), March 2021); spatial interplay of tumor-infiltrating lymphocytes (SpaTIL; see, e.g., Corredor, G. et al., “Spatial Architecture and Arrangement of Tumor-Infiltrating Lymphocytes for Predicting Likelihood of Recurrence in Early Stage Non-Small Cell Lung Cancer,” Clin. Cancer Res. 25(5), March 2019); variations on SpaTIL for gynecologic cancers (ARCTIL; see, e.g., Azarianpour, S. et al., “Computational image features of immune architecture is associated with clinical benefit and survival in gynecological cancers across treatment modalities,” J. Immunother. Cancer 10(2), February 2022); variations on SpaTIL for oropharyngeal cancers (OP-TIL; see, e.g., Corredor, G. et al., “An imaging biomarker of tumor-infiltrating lymphocytes to risk-stratify patients with HPV-associated oropharyngeal cancer,” J. Natl. Cancer Inst. 114(4), pp. 609-617, April 2022); variations on SpaTIL for patients who have received immunotherapy (Histo-TIL; see, e.g., Wang, X. et al., “Spatial interplay patterns of cancer nuclei and tumor-infiltrating lymphocytes (TILs) predict clinical benefit for immune checkpoint inhibitors,” Science Advances 8(22), June 2022); and variations on SpaTIL that quantify TIL sub-populations and the interplay between these populations (PhenoTIL; see, e.g., Barrera, C. et al., “Phenotyping tumor infiltrating lymphocytes (PhenoTIL) on H&E tissue images: predicting recurrence in lung cancer,” Proc. SPIE 10956, Medical Imaging 2019: Digital Pathology 1095607, May 2019).

In extracting features in task 16, the set of features that is extracted will often be predefined based on the type of biomarker or assay that is to be established. The pre-defined set of features may include any combination of vessel morphology features and other radiomic and pathomic features. As will be described below in more detail, in a method like method 10, where the ultimate objective is to establish a biomarker or assay, it is usually advantageous to extract as many potentially relevant or determinative features as possible, and in implementing method 10, it may not be known in advance whether a particular feature or type of features is relevant.

Depending on the particular features that are extracted, task 16 may also involve normalization or other processing of features post-extraction. As those of skill in the art will understand, it is possible that one or more features may not be available for a particular patient in a particular data set. If that is the case, it may still be possible to use that patient's data by filling any missing features with the median value for that feature, or by using another, similar type of value.

In one example, if only vessel morphology features are used, several hundred distinct features (e.g., 100-600 features) may be extracted from a three-dimensional vessel segmentation of lesion-associated vasculature and used in following tasks of method 10.

In another example, an embodiment may use several hundred distinct vessel morphology features, along with sets of radiomic texture features extracted from the area of the lesion, and optionally, texture features extracted from the peri-lesional area, i.e., the area around the lesion. A peri-lesional area can be defined either by morphological dilation of the boundaries of a lesion by a specific distance, or, e.g., by drawing a boundary some distance from a centroid of the lesion.

In yet another example, an embodiment may use vessel morphology features, intra-lesional and peri-lesional radiomic texture features, and a set of pathomic features extracted from a WSI of tissue taken from the lesion.

Once a useable set of extracted features is available, method 10 continues with task 18.

Task 18 is an optional task. As was described above, a biomarker according to an embodiment of the invention may be based on vessel morphology features alone, or it may be based on a combination of vessel morphology features with other radiomic and/or pathomic features. Additionally, a biomarker may be based on a combination of features and other types of patient data. In task 18, if other patient data is to be used as a part of a biomarker, that other patient data is acquired.

Other patient data may include demographic data (e.g., age, gender, race/ethnicity, height, weight, sexual orientation); medical history data (e.g., current or previous diagnoses, staging of any cancers, previous treatments); general biochemical and metabolic data (e.g., data from a complete blood count (CBC), comprehensive metabolic panel (CMP), and other tests of blood, urine, cerebrospinal fluid, etc.); test data specific to particular diagnoses (e.g., programmed cell death-ligand 1 (PD-L1) expression testing, human epidermal growth factor receptor 2 (HER2) status, etc.); and any other patient data typically stored in a medical record. If such data is to be used, it may be acquired from an electronic medical record (EMR) system in task 18.

In some cases, other patient data extracted in task 18 may need to be transformed or normalized in some way so that it can be used in a method like method 10. That may also be done in task 18 in the process of acquiring the data. For example, the acquired data may be binarized in various ways (e.g., a patient's race/ethnicity may be rendered as Caucasian or non-Caucasian, a patient's height and weight may be processed to a simple indication of whether the patient is considered to be medically obese, and tests specific to particular diagnoses may be transformed from raw numerical values using thresholds or other techniques so that a test result is reported as either positive or negative). Method 10 continues with task 20.

In task 20, relevant phenotypes are established. This may be performed, e.g., by using a machine model to cluster the patients according to the features extracted from each. The type of clustering that is performed may vary from embodiment to embodiment, but as one example, unsupervised k-means clustering may be used to establish phenotypes, with each cluster being a phenotype.

K-means clustering may be useful when there is a known quantity of expected phenotypes. Other unsupervised clustering techniques may be used. For example, a process of collaborative clustering may be used when there are more than two expected phenotypes, or when it is desirable to explore how many distinct phenotypes there are based on the extracted features. Collaborative or consensus clustering is described in, e.g., Monti, et al., “Consensus Clustering: A Resampling-Based Method for Class Discovery and Visualization of Gene Expression Microarray Data,” Machine Learning 52, pp. 91-118 (2003).

Briefly, the consensus clustering process begins by defining a minimum and maximum number of clusters (e.g., K=2 to 10). For each of the proposed number of clusters (i.e., for each value of K), the original feature matrix is subsampled, and a clustering algorithm is executed to group the subsampled data into K unique clusters. Once those unique clusters are created, samples that are clustered together and samples that are in different clusters are identified. Those basic steps, subsampling, clustering, and identifying which samples are clustered together, are repeated a number of iterations while a consensus matrix is assembled that tracks how often two samples are clustered together across all iterations. A consensus score for K is then calculated based on the consensus matrix.

After clusters and consensus scores are established, the process continues with choosing the optimal number of clusters by evaluating the stability of the clusters. This can be done by considering or visualizing the cumulative distribution function (CDF) of the consensus scores, the area under the CDF (AUC), and the change in the AUC, among other metrics. For example, the change in AUC may indicate that the optimal number of clusters is two. In some cases, the metrics may differ, for example, if the largest change in the AUC happens with K=2, but a graph of the AUC vs. the number of clusters shows that the AUC begins to “level off” at K=5. In that case, the number of clusters that makes the clearest distinctions between the clusters may be used.

Of course, the above describes two unsupervised machine learning techniques for establishing the clusters, and thus, the phenotypes. Supervised learning techniques could also be used. For example, a machine learning model, and in particular, a machine learning classifier, such as Least Absolute Shrinkage and Selection Operator (LASSO), could be trained to identify the phenotypes based on the extracted features. Supervised machine learning may be particularly helpful when one is attempting to replicate existing biomarkers and phenotypes, especially for clusters discovered using unsupervised learning techniques that are not easily applied to new data (for instance, consensus clustering).

Method 10 continues with task 22.

Once phenotypes are established, method 10 continues by associating those phenotypes with relevant phenomena or outcomes. The manner in which this is done will depend on the phenomena or outcomes that are of interest. In various embodiments, this may involve calculating, for each cluster or phenotype, one or more of: likelihood of a particular diagnosis, likelihood of response to a particular treatment, likelihood of recurrence given a particular treatment, overall survival, disease-free survival, progression-free survival, etc. Essentially any phenomenon or outcome for which data exists can be calculated for each phenotype. For example, based solely on vessel morphology features, it may be established which phenotype(s) are more likely to respond to a VEGF/PD-1 bispecific antibody like ivonescimab, to another type of bispecific or multi-specific treatment that targets angiogenesis in part, or to a purely anti-angiogenic treatment, like bevacizumab. For example, a high-risk phenotype that responds more poorly to immune checkpoint inhibitors may benefit from escalating therapy to include anti-angiogenics. As another example, based on a vessel morphology features alone or a combination of vessel morphology features with one or both of other features or other patient data, it may be established which phenotypes are more likely to respond to an immune checkpoint inhibitor (ICI) like pembrolizumab, ipilimumab, nivolumab, or atezolizumab. As to outcomes, task 22 may involve determining which of the phenotypes are more likely to experience greater overall survival, progression-free survival, disease-free survival, or recurrence.

In associating phenotypes with relevant phenomena or outcomes, it may be desirable to use regression analysis or other known tools to explore the outcome related to a single variable or characteristic, independent of others. For example, while patient age, gender, histology, and the presence of other biomarkers may be considered, it may be useful to consider the effect of only one variable, independent of the others. As a further example, it may be helpful to consider the impact of vessel morphology on a phenomenon or outcome of interest, like overall survival or response to a particular treatment, independent of other variables.

Method 10 continues with task 24, in which the clusters or groups with associated outcomes may be further processed for use as biomarkers and phenotypes. This task may not be necessary in all embodiments—that is, once a particular cluster or group within the cohort is associated with an outcome, that may be all that is necessary to define a biomarker and associated phenotype. To determine whether a patient exhibits a particular biomarker, the features used to establish the appropriate phenotype may simply be extracted and classified, as will be described below in more detail. However, after determining that a particular phenotype—established with a particular set of features—is useful as a biomarker, it may be desirable to use feature selection techniques to establish the minimum set of features necessary to define the particular phenotype. While not strictly necessary in all embodiments, establishing the minimum set of features necessary to define a particular phenotype may simplify computation and reduce the time and resources necessary to assay for biomarkers in research and clinical practice. Feature selection techniques may include, but are not limited to, the minimum redundancy, maximum relevance (mRMR) approach, principal component analysis, and the like.

Additionally, irrespective of whether a supervised or an unsupervised clustering technique was used in task 20, in task 24, a machine learning model can be trained to predict the clusters and to assess the top features driving the clusters, as well as their directionality. For example, a random forest model can be trained to predict the clusters and to provide a listing of, e.g., the top 10, 20, 30, 50, etc. features. If desired, after establishing the top features driving the clusters, or after other techniques to remove highly-correlated features, method 10 may return to task 20, biomarkers and associated phenotypes may be re-established with the reduced feature set, and the performance of the biomarkers and phenotypes established with the full feature set may be compared to the performance of the biomarkers and phenotypes established with the reduced feature set.

Method 10 concludes at task 26.

### Assaying for Biomarkers

Once a biomarker and associated phenotype is established, it can be used in research and in clinical practice. As those of skill in the art will note, the medical images used to extract features in methods and systems according to embodiments of the invention are generally routine clinical images, i.e., medical images acquired in the course of routine clinical tasks like diagnosis and treatment, rather than medical images acquired specifically for use in methods according to embodiments of the invention. This means that it may be possible to assay for the presence of a particular biomarker or biomarkers based on, e.g., a single clinical CT or MRI scan. A biomarker assay may be performed automatically in some cases, as soon as a suitable scan is entered into an RIS or a patient's EMR. However, an assay may also be ordered by a researcher or clinician like any other laboratory test. In general, the assays described here may also be ordered at the same time and in the same manner as any of a number of genomic assays used to classify diagnosed cancers and to make predictions as to recurrence. For example, any patient criteria specific to the particular assay may be eligible for a treating physician or a researcher to order or request an assay.

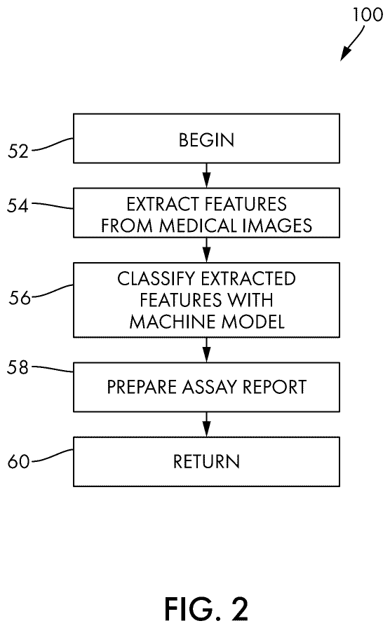

FIG. 2 is an illustration of a method, generally indicated at 50, for performing an assay for the type of biomarker established in a method like method 10. The initial tasks of method 50 proceed much like those of method 10 above.

More specifically, method 50 begins at 52 and continues with task 54. In task 54, features are extracted from medical images, such as medical images derived from a CT or MRI scan. As in method 10, preparatory tasks may be necessary to prepare the medical images before features can be extracted. For the sake of simplicity, a description of those tasks is not repeated here. In task 54, it is assumed that a three-dimensional vessel segmentation is available for vessel morphology extraction and, if other features are used, that the medical images are prepared for feature extraction. For simplicity, this description assumes that if any other patient data is used, that data is acquired in task 54.

In contrast to method 10, in extracting features in task 54, typically, only those features known to be a part of the desired biomarker, or known to be necessary for the desired purpose, are extracted. For example, if a biomarker is based only on particular vessel morphology features, only those particular vessel morphology features are extracted. Method 50 continues with task 56.

In task 56, a model is used to classify the extracted features and, thereby, to assay for the presence of the biomarker. In virtually all cases, the model will be a machine learning model. The machine learning model may, e.g., be a classifier trained to distinguish patients who have the biomarker from those who do not based on the sets of features extracted in task 54. The classifier may be, e.g., a logistic regression or Cox proportional hazards model, a linear discriminant analysis (LDA) classifier, a quadratic discriminant analysis (QDA) classifier, a bagging classifier, a random forest classifier, a support vector machine (SVM) classifier, a Bayesian classifier, a LASSO classifier, etc. A trained neural network may also serve as a classifier.

Method 50 continues with task 58. In most cases, the output from the machine learning model in task 56 is a probability or risk score. In some embodiments, this score may be sufficient. Task 58 is an optional task in which the result of task 56 is rendered more interpretable. This task may be simple or complex. For example, in a simple implementation, a threshold may be applied to a raw probability score so that the result can be rendered as “positive” or “negative” for the biomarker. In other cases, task 58 may involve the use of either a simple algorithm or a generative machine learning model that takes the raw probability score and produces a textual or graphical output that is more interpretable to clinicians and researchers.

For example, in some cases, the result of method 50 may be a report that includes the biomarker result, a generated explanatory text, selected exemplary vessel morphology results, and graphical depictions of exemplary portions of the vessel morphology.

Method 50 terminates at task 60.

Method 50 is a simple example of how a biomarker established with a method like method 10 might be used. It might provide, for example, a simple assay for a biomarker, resulting in a “positive” or “negative,” optionally with some generated explanatory material to ease interpretation. Researchers and clinicians can then predict the effect of that result using clinical judgment and experience or a simple model, like a nomogram.

However, a classifier is not the only type of machine learning model that may be used in embodiments of the invention. As another example, U.S. patent application Ser. No. 18/786,417, filed Jul. 26, 2024, the contents of which are incorporated by reference herein in their entirety, discloses an oncological foundation model that uses a transformer-based architecture. As the '417 application discloses, this foundation model is adapted to take extracted features, as well as whole medical images or complete scans, as input. Such a model could be used to determine whether a biomarker is present given a set of features and then make more complex sorts of predictions. For example, such a model could be used to predict not only response to a particular treatment, overall survival, or progression-free survival, but also the longitudinal course of a patient's disease, in some cases, predicting how scans are likely to appear at given points in times. Such models may also be able to provide comparative or counterfactual predictions, predicting how a patient will fare when given one treatment as opposed to another.

As may be apparent from the above, methods like method 10 and method 50 may be used in all stages of treatment and for research applications. Diagnostically, the kinds of biomarkers described here may be useful in determining whether a particular lesion is benign or a malignant tumor, and what type of lesion or tumor. In pre-treatment, clinicians may assay for the kinds of image-based biomarkers described here to determine who will respond to a particular treatment, such as immunotherapy or a combined VEGF/PD-1 treatment. Biomarkers may also be used to evaluate treatment strategy—e.g., which patients are likely to respond to immunotherapy alone, immunotherapy plus chemotherapy, etc. In some cases, if a specific biomarker is identified, that biomarker may be used to select the particular treatment agent, or, in a broader context, to define first-line and second-line treatment options in patient populations having particular biomarkers.

Methods 10 and 50 describe the creation and use of a single biomarker, and phenotypes and assays based on that biomarker. Working embodiments of the invention may use multiple biomarkers to make predictions. For example, one biomarker may be established based solely on vessel morphology features, and a second biomarker may be established based on vessel morphology features with added intra-lesional and peri-lesional texture features. A third biomarker may be established based on radiomic vessel morphology features, radiomic texture features, and pathomic features. Those biomarkers may be used, individually and/or collectively, to predict, e.g., the probability of recurrence in NSCLC given a particular treatment, or the probability of recurrence in NSCLC given any of a number of treatment options.

Although portions of this description focus on NSCLC, the methods described here may be used with any number of types of lesions to establish their nature and a diagnosis, and with any number of diagnosed tumors, including lung cancer, breast cancer, ovarian cancer, colon cancer, oropharyngeal cancer, pancreatic cancer, etc. As was noted above, the methods and systems described here may also be used in the course of determining the efficacy of a drug or other treatment in a clinical trial.

As may be apparent from the above description, some of the features described here are sub-visual, and others, while they may be appreciated visually, are calculated in enormous quantities, e.g., hundreds of features at tens, hundreds, or thousands of different locations. Thus, a working embodiment of method 10 or method 50 cannot be performed in the mind or with pen and paper. In general, radiomics and pathomics cannot be practiced in the mind or with pen and paper. As those of skill in the art will appreciate, working embodiments of methods 10 and 50 provide an improvement in the ability of a computer, and specifically, a trained machine learning model, to make predictions concerning lesions, and ultimately, an improvement in the ability of a computer to help researchers and clinicians explore and manage cancers. Part of that improvement is greater interpretability of results generated with machine learning.

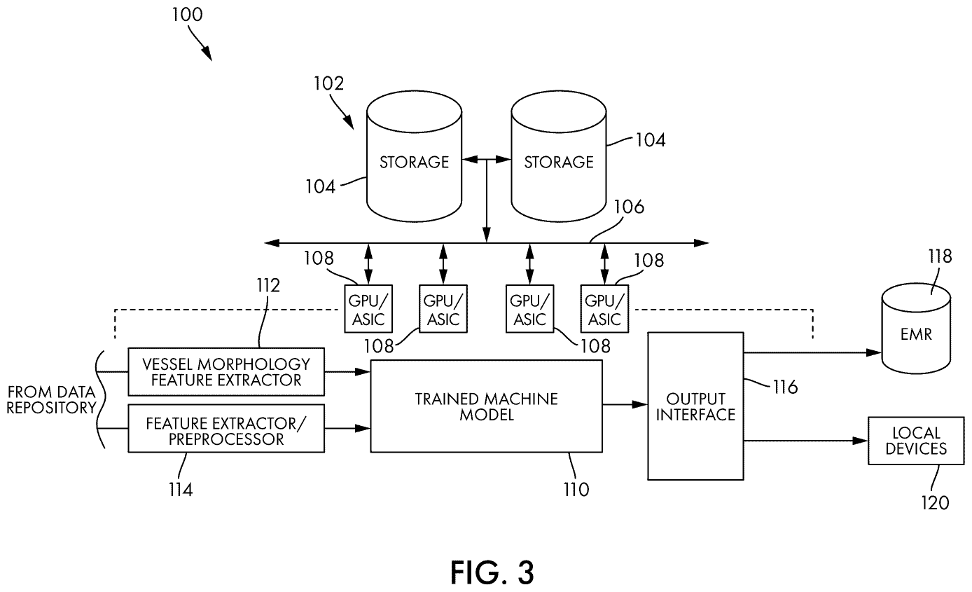

FIG. 3 is a schematic diagram of a system, generally indicated at 100, for implementing methods according to embodiments of the invention, including methods 10 and 50. System 100 is typically implemented using a cloud computing system 102, i.e., a large-scale system physically implemented in a data center or other dedicated facility that is used through a computer network, such as the Internet. The cloud computing system 102 generally includes one or more storage devices 104, a data bus 106 or other hardware for interconnecting components, and one or more processors 108. A typical cloud computing system 102 includes numerous processors 108, numerous storage devices 104, etc., all connected together and in communication with one another. The processors 108 may be general-use microprocessors, but in many embodiments, the processors 108 will be computer processing elements that are more capable or more specialized for machine learning use, like graphics processing units (GPUs), or application-specific integrated circuits (ASICs), like tensor processing units (TPUs). The cloud computing system 102 would typically also be equipped with memory, such as random-access memory (RAM) and read-only memory (ROM), although for simplicity, these are not shown in FIG. 3.

The other components shown within the cloud computing system 102 in FIG. 3 are software modules. That is, they are comprised of machine-readable instructions (i.e., software code) on machine-readable media that, when executed by machines like the processors 108 and their connected components, cause the processors 108 to perform the functions described here. The machine-readable instructions are typically stored on the storage devices 104, although while the processors 108 are executing the instructions, instructions may be stored in a temporary form of memory, like RAM or an on-processor cache.

Although a cloud computing system 102 is described here and shown in FIG. 3, that does not necessarily preclude system 100 from being installed in a particular location for use at that location (i.e., an on-site installation). System 100 could, in at least some embodiments, be installed at a single location (e.g., a hospital) for use at that location. However, as those of the art will understand, the amount of computing hardware necessary, the amount of space required, and other considerations, like power consumption, generally make it more convenient for systems like system 100 to be cloud-based. (That is, in practical terms, housed in a dedicated data center and accessible through the Internet or a dedicated wide- or local-area network.)

At the core of the software modules of system 100 is a trained machine learning model 110 or models, which may be of any of the types described above. The machine learning model 110 is specifically trained to accept and to make a prediction or predictions based on specific types of features. In this description, those specific types of features include at least a set of vessel morphology features, and in various embodiments, may also include various radiomic and pathomic features, as well as other patient information.

In this embodiment of system 100, a number of feature extractor/preprocessors 112, 114 that are specialized for different types of features. For example, the feature extractor/preprocessor 112 is adapted to perform prefatory tasks and to extract vessel morphology features. This module may create the three-dimensional segmentation of the vasculature and perform other prefatory tasks before extracting the pre-defined set of vessel morphology features. Similarly, the feature extractor/preprocessor 114 is adapted to preprocess and extract some other type of feature (e.g., other radiomic features, pathomic features, etc.). System 100 may have any number of feature extractor/preprocessors 112, 114, each adapted for a different type of feature, or it may have none at all, e.g., if preprocessing and feature extraction are done on other systems, i.e., using other equipment.

The prediction or predictions made by the machine model 110 may be sent to an output interface 116, which may further process the prediction to aid in interpretation or to allow the prediction to be communicated to various other systems, like an EMR 118 and local devices 120, like local computers, tablets, and smart phones. In a practical implementation of system 100, there may be several output interfaces 116, each with a specialized function. For example, one output interface 116 or interface module may be a generative machine learning model that focuses on producing an interpretable assay report from the predictive result generated by the machine model 110, while other output interface modules 116 focus on formatting the result as necessary for entry into various external databases and systems 118, 120. In some cases, an output interface 116 may have a module that acts as a web server and instantiates a graphical interface.

Embodiments of the invention may also include EMR systems 118 that have user interfaces allowing a user, such as a researcher or a clinician, to order an assay for a biomarker such as the biomarkers described above, to view the results of such an assay, to classify the patient as having one particular phenotype or another based on the biomarker(s) or assay(s), or to view predictions or reports made or based on the kinds of biomarkers described above. As was noted briefly above, such assays may be ordered in the same ways as traditional laboratory and genetic tests.

### Changes in Phenotype as a Predictor

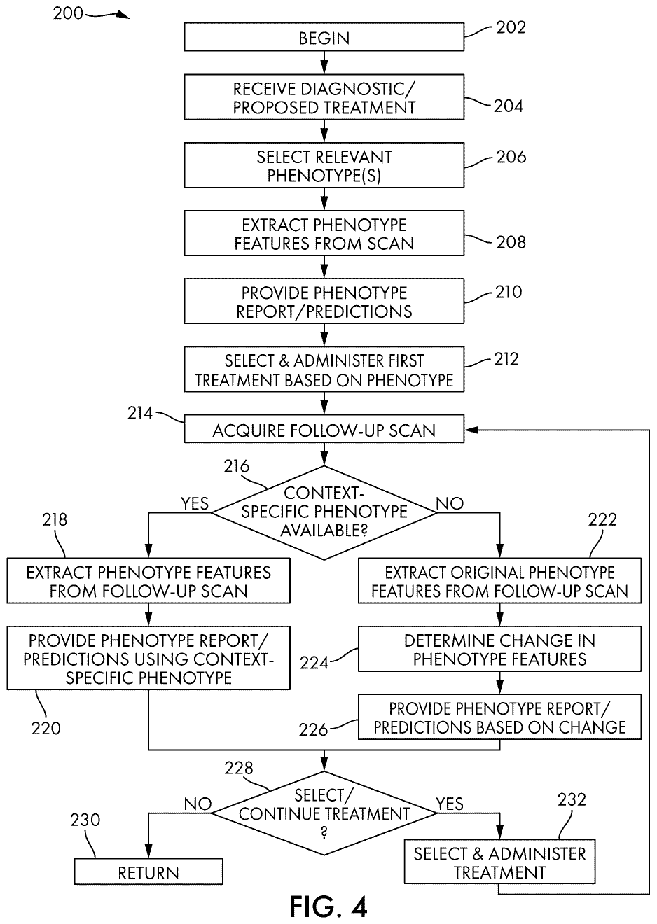

Radiomic vessel morphology and combined-feature phenotypes may be used at multiple points in time, throughout the diagnostic and treatment process. FIG. 4 is a flow diagram of a method, generally indicated at 200, for using phenotypes throughout a treatment process.

One goal of methods like method 200, and the use of the kinds of phenotypes, biomarkers, and assays described here, is to treat a patient with the most effective treatment as early in the treatment process as possible. This means identifying, as early as possible, whether a patient is responding to a treatment and, if not, moving that patient to another treatment to which there is a higher probability of response as soon as possible. Phenotypes, and biomarkers based on those phenotypes, may be able to accomplish this at earlier stages and with a higher probability of success than traditional metrics, like the Response Evaluation Criteria for Solid Tumors (RECIST). For this reason, methods like method 200 may be used in clinical practice, in research, and in clinical trials, among other settings.

For purposes of explanation, method 200 assumes that the patient has not been given any treatment, i.e., that method 200 begins just after diagnosis with the patient “naïve” to treatment. However, the description provided here is by way of example only; method 200 and other methods like it may be begun and used at any point in the diagnostic or treatment process.

Method 200 begins at 202 and continues with task 204. At task 204, it is assumed that a medical provider, or another such party, has ordered an analysis of a patient's scans to find radiomic vessel morphology phenotypes and biomarkers. Thus, task 204 begins by receiving any relevant information on the patient, including demographic information, diagnosis and staging information, information on the proposed treatment or treatments, the presence of genetic or histochemical biomarkers, etc. In some cases, this may be done automatically by an EMR system 118 when a biomarker assay is ordered. In other cases, a medical provider may select certain information to be shared. Method 200 continues with task 206.

In task 206, a system like system 100 identifies a biomarker or biomarkers that are relevant to the patient or to the medical situation. In some embodiments, this may be a single biomarker; in other embodiments, there may be several biomarkers relevant to the patient or to the medical situation. Of course, as those of skill in the art will realize, tasks 204 and 206 may be optional. That is, in some embodiments, there may be a single biomarker for which each patient is assayed, making patient information and biomarker selection unnecessary.

Once the relevant biomarker or biomarkers are identified, method 200 continues with task 208, and the relevant features for the associated phenotypes are extracted from the patient's radiology scans. These features will generally comprise at least a set of radiomic features related to vessel morphology and, as described earlier, may comprise other radiomic features, pathomic features, and other types of data. Extraction will typically be conducted as described above, although in task 208, the features extracted may be limited to those that are a part of a phenotype known to serve as a biomarker or otherwise known to be relevant. Method 200 continues with task 210.

In task 210, the extracted features are processed to determine whether the tumor belongs to a pre-established phenotypical group. This can be done using machine learning techniques in a number of supervised and unsupervised ways, including by clustering (e.g., k-means or collaborative clustering), by using a pre-trained machine learning model to classify the extracted feature set as belonging to a pre-established phenotypical group or not, etc.

While the output of task 210 is usually at least an indication of whether or not a particular tumor belongs to a particular pre-established phenotypical group based on the extracted features, the output may include significantly more. For example, there may be cases in which the extracted features demonstrate that while a particular tumor does not fall within the precise bounds of a particular phenotypical group, it is closer to one phenotypical group than to another. These finer gradations in tumor characteristics may be clinically relevant.

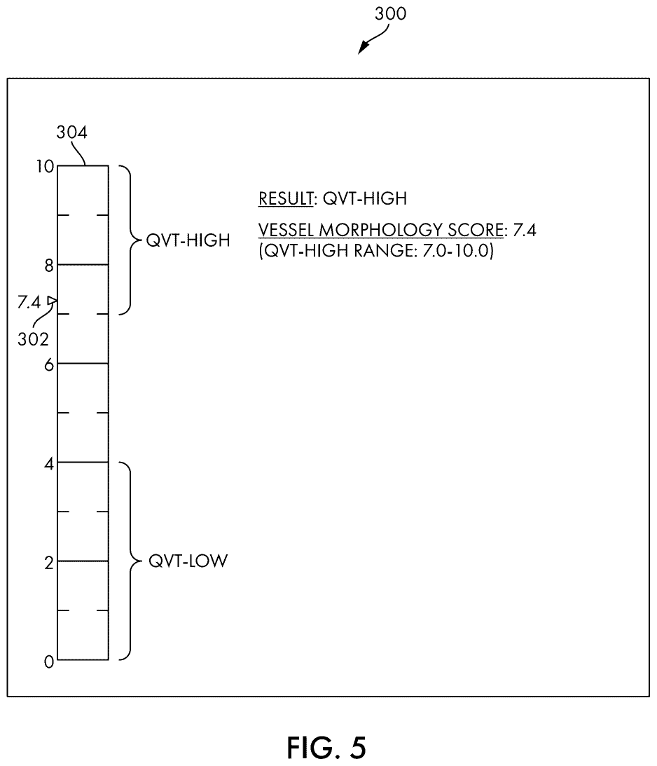

For those reasons, the output of task 210 may be a continuous score that allows a practitioner to assess the overall vessel morphology of the tumor relative to the known phenotypical groups. FIG. 5 is an illustration of an output report, generally indicated at 300, that includes a continuous score. In this report, the score, indicated at 302, is shown against a reference range 304 that indicates the score ranges for two pre-established phenotypical groups, QVT-high and QVT-low. As with many other types of medical tests and reports, the output report 300 with its reference range 304 provides the interpreter with more context than a simple yes/no or indication of a phenotype might. In some cases, if the outcomes support it, the reference range 304 could be reported with a green-to-red color gradient, or some other type of gradient to indicate “good” to “bad.” However, this description does not assume that a higher score is necessarily worse, or that a lower score is necessarily better—that is context-dependent.

The score itself may be arrived at using a number of different techniques. In establishing a score, the underlying features may all be given the same weight, or they may be weighted or normed depending on their contribution to the phenotype determination. (That is, the most determinative features could be weighted more or less.) The scale of the score may be linear, logarithmic, or any other suitable scale.

Once task 210 is complete, method 200 continues with task 212, in which a treatment is selected and administered based on the phenotype. In some cases, a report like the output report 300 of task 210 may contain explicit treatment recommendations, such as “likely to respond to an immune checkpoint inhibitor” or “likely to respond to combined anti-angiogenic and immunotherapeutic agents” that are derived based on the vessel morphology score or other aspects of the results of task 210. In other embodiments, there may be no explicit treatment recommendation, and the selection of a treatment in task 212 may be left to clinical judgment, based on the phenotype or vessel morphology score. At some point after a treatment is administered, a follow-up scan will typically be performed, as shown in task 214.

As those of skill in the art will note, to the point of at least task 212, method 200 resembles the kind of assaying method described above. However, in method 200, phenotypes are used at multiple points in the diagnostic and treatment process. The phenotypes that are used at each stage in method 200 may be the same or different.

More specifically, some phenotypes and biomarkers based on them may be relevant throughout the treatment process. These will usually be those phenotypes constructed using broad data. In addition, as a patient moves through treatment, new phenotypes, relevant to particular contexts and particular treatments, may emerge. Methods like method 200 may use any type of phenotype.

Thus, task 216 is one example of the kind of decision task that could be used to determine which phenotype(s) should be applied in later stages of analysis and treatment. In task 216, if there is a phenotype or set of phenotypes that is specific to the present context (e.g., specific to the treatment or treatments that the patient received) (task 216: YES), then method 200 continues with task 218, features specific to that new phenotype are extracted from the follow-up scan, and a report or other output is provided in task 220. The report provided in task 220 may be similar to the output report 300 of FIG. 5, although it may include other context-specific information.

Context-specific phenotypes may be helpful in making specific kinds of predictions and judgments in particular situations, like whether a patient, having been given a first-line treatment, is likely to respond better to another treatment. However, as noted above, depending on the breadth of the data used to establish a phenotype, that phenotype may be generalizable to a variety of different situations. If the original phenotype used in tasks 208-210 of method 200 is broadly generalizable, if no context-specific phenotype is available, or if it is desirable to use the original phenotype or set of phenotypes (task 216: NO), method 200 continues with task 222.

In task 222, the features that comprise the original phenotype are extracted from the follow-up scan taken in task 214. Method 200 then continues with task 224, and the change between the original phenotype result and the present phenotype results is determined.

Change may be determined in various ways, including both direct and indirect measures of change. Direct measurement of change may involve, e.g., directly calculating the change between all newly-extracted feature values and all previous feature values extracted earlier in method 200. However, it may not be necessary to determine the change in each feature value. One could, for example, measure change in only the features known to be most determinative of phenotype, e.g., the top 5 features, the top 10 features, the top 20 features, the top 50 features, etc.

Indirect measurement and determination of change may involve comparing an initial composite score, like the vessel morphology score described above with respect to FIG. 5, with a similar score established based on a later scan. Other types of normalized, aggregate measures of features may also be used in task 224 of method 200. Such indirect comparisons generally do not involve direct comparison of one feature value to another, but they may be more convenient, faster to implement, and likely to provide the necessary information. Whether one uses a direct measurement/comparison strategy or an indirect one in task 224 may depend on the particular circumstances. For example, in clinical practice with well-established phenotypes, an indirect comparison of, e.g., one composite score with another may be sufficient to provide the necessary information for treatment purposes. By contrast, in a clinical trial or other investigational or research situation, it may be more desirable to catalog the changes in individual features, so as to understand not only the phenotypical impact of a treatment but also the underlying changes in vessel morphology, tumor kinetics, etc. at a deeper level.

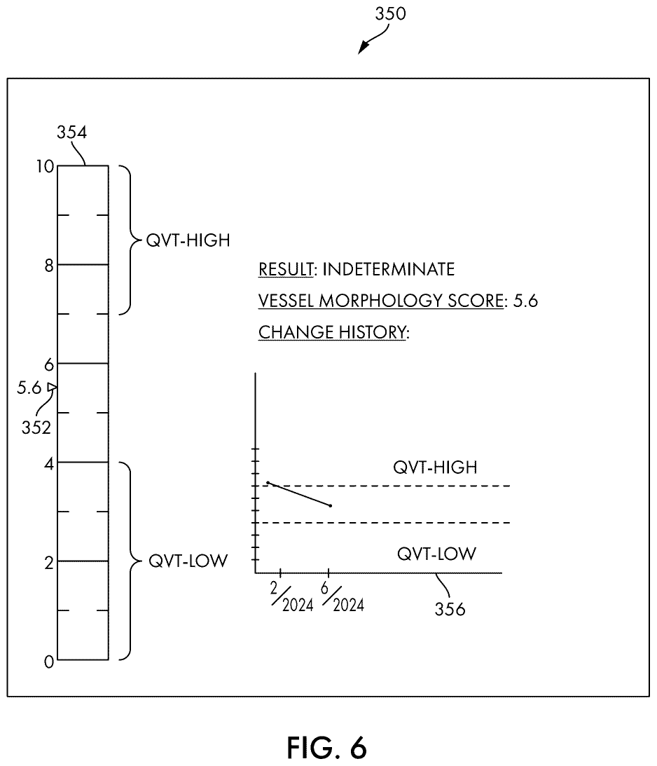

Once any determination or assessment of phenotypical change is complete in task 224, method 200 proceeds to task 226, and some type of report or output is provided indicating the result. FIG. 6 is an example of a report, generally indicated at 350, that continues the example and conventions of FIG. 5. FIG. 6 assumes that the same phenotype has been used at each stage in method 200 (in other words, there was no context-specific phenotype to use in task 216). In the report 350, a score 352 is again shown against the same or a similar reference range 354. In this example, the vessel morphology score has decreased from a 7.4 in the example of FIG. 5 (i.e., task 210) to a 5.6. Because the minimum score for the QVT-HIGH phenotype in this example is 7.0 and the maximum score for the QVT-LOW phenotype in this example is 4.0, the result is indeterminate, and the report 350 states that. Also included is a graph 356 that tracks the change in vessel morphology score over time. The graph 356 shows, in particular, the dates of the two scans and the decline in vessel morphology score.

As the report 350 of FIG. 6 illustrates, the results of a biomarker assay (i.e., an assay of phenotype), need not be binary. Moreover, even if the result of an assay is indeterminate with respect to particular phenotypes, the result may still provide clinically useful information. How the information is used will depend on the clinical context. For example, if the phenotypical group does not change, that may suggest no change in treatment plan. However, if the report 350 does show change in the desired direction (in this case, at least some decrease in chaotic vessel morphology), other factors may be considered in a treatment decision. The vessel morphology could be one variable in a clinical nomogram that provides a heuristic for decision-making based on multiple pieces of evidence. For example, if a vessel morphology result is indeterminate, a clinician might look at the size of the tumor and other such traditional variables to make an overall decision.

The reports 300, 350 of FIGS. 5-6 show the vessel morphology score presented alone. However, vessel morphology scores may be presented with, used with, or even combined with, other types of phenotypical scores and metrics. For example, a size change score based on changes in the size of the tumor, a body composition score derived from the composition of the patient's body, and pathomics-based scores may all be used in conjunction with the types of vessel morphology scores disclosed here.

If no further treatment is required (task 228: NO), method 200 returns at task 230. If a further treatment is required or desired, that treatment is selected and administered in task 232 before method 200 returns to task 214 and continues from that point. In other words, as treatments continue, phenotypes, biomarkers based on those phenotypes, and assays for those biomarkers, continue to be useful tools in predicting response and choosing the best treatment for each individual patient throughout the process.

Many variations on the basic scheme of method 200 are possible. For example, while the later tasks of method 200 describe selecting either a context-specific phenotype or a more general phenotype for use, in at least some cases, both may be used at the same time. That is, a general phenotype used at the outset of treatment may be used in later stages of treatment in addition to context-sensitive phenotypes. One might, for example, determine the change in a general phenotype that was used previously while also determining whether the patient now fits into particular context-specific phenotypical groups.

Additionally, while the above description focuses on understanding the amount of change between a pre-treatment phenotyping and a post-treatment phenotyping, the rate of change could also be used as a relevant variable in clinical and research decision-making. In that case, for example, a treatment that caused a rapid, desirable change in a patient's tumor vessel morphology might be viewed more favorably and continued even if the phenotypical results per se would not support continued treatment.

### Additional Radiomic Vessel Morphology Features

The above description provides certain examples of vessel tortuosity features. Those features are only one type or family of vessel morphology features. This section provides additional examples.

One additional type or family of features comprises vessel torsion features. In general, the term “torsion” refers to the twisting of a blood vessel around its longitudinal axis. Torsion can be quantified using the Frenet-Serret frame, a coordinate system that is attached to a curve at each point. The Frenet-Serret frame consists of three vectors: the tangent vector T, which points in the direction of the curve; the normal vector N, which points perpendicular to the curve in the plane spanned by the tangent vector and the binormal vector B, and the binormal vector B, which is perpendicular to both the tangent vector and the normal vector. Within this frame of reference, torsion is defined as the rate of change of the binormal vector B with respect to the arc length of the curve. Alternatively, torsion can be viewed as the amount of twisting per unit distance. As with other features in this description, torsion can be measured globally over the entire segmented vasculature, by branch, or over some other portion of the vasculature.

To calculate torsion in feature extraction methods, the segmented vasculature would be processed to identify vessel centerlines and to fill in gaps. Following those prefatory steps, which may be done at any time before feature extraction, one would compute the T, N, and B vectors, and from them, the torsion. The resulting torsion values may be either positive or negative, indicating the direction of the torsion. For that reason, the absolute value of the torsion or magnitude of the torsion may be considered to be another feature.

Some features may be derived from torsion measurements, or from the T, N, and B vectors used to calculate torsion. For example, the text above describes the use of inflection points, places where the direction or sense of the vessel curvature changes. A larger number of inflection points per unit of vessel length may indicate that the vessel is more twisted. Inflection points can be found in a number of ways, including, as described above, by looking at the sense of the vessel tortuosity. However, inflection points can also be calculated using the vessel torsion. To do so, one would first compute the torsion along a length of vessel, and then select all points at which the magnitude of the torsion is over a threshold. The sign or sense of the torsion at those points are examined, and an inflection point is identified between points of opposite sign.

Inflection point features may also be considered to be a family of radiomic features. Features such as the number of inflection points per unit length of vessel, the number of inflection points per vasculature branch, and the number of inflection points per some other portion of the vasculature may all be used. In other words, as with other features, inflection point features may be considered both globally—across the entire vasculature—and locally, in a branch or in individual vessels.

Other features may also be derived from the Frenet-Serret frame vectors T, N, and B. For example, it may be helpful to establish the gradient of the torsion and the gradient of the vessel curvature toward (or away from) the lesion. This feature describes, essentially, how the torsion and curvature change along the length of a vessel, and may indicate regions where there are rapid changes.

The torsion-to-curvature ratio can also be calculated and used as a feature. This may indicate areas where twisting dominates over bending, or vice-versa. If twisting and bending are highly correlated, which they may be in at least some cases, then areas where they are not highly correlated (i.e., areas where the ratio is particularly high or particularly low) may be predictive in at least some applications.

The torsion-curvature product may be calculated by multiplying the torsion and curvature at a particular point and used to indicate areas where twisting and bending occur simultaneously. These areas, which may be considered to be areas of complex vessel dynamics, may be predictive in at least some applications.

Another vessel morphology feature that may be extracted is the ratio of the length of a vessel along the curve compared with the straight-line distance from one endpoint to another endpoint. As with the features described above with respect to method 10, this feature may be taken over various spans of points placed along the vessel, and the “window” of points may be expanded or moved along the vessel. For example, for granular results, this feature may initially be established every 5 points, every 6 points, etc.

The above features primarily describe the magnitude of the torsion and curvature, but the direction or “sense” of the torsion and/or curvature, and the way in which that direction or sense changes along the length of a vessel, may also be useful descriptive features. For this reason, variation in the normal and binormal vectors N, B may be calculated and used as features. This may, for example, involve taking the derivatives of these vectors N, B. The vector angles may also be directly computed, and the change in those angles may be extracted and used as a feature or features.

Measures of variation of a vessel's direction may also be used. For example, for any given segment of a vessel with two defined endpoints, one can choose a point along the vessel between those two endpoints and extract the straight-line angle formed between the chosen point and the endpoints. The endpoints may be selected arbitrarily and, in some cases, moved progressively along the vessel.

There are other ways of characterizing the morphology of the vasculature. For example, it is possible to consider the physical or mechanical properties inherent in the morphology of a vessel, rather than the morphology itself, and to use those properties as radiomic vessel morphology features. More specifically, a curved or twisted object, like a vessel, can be considered to have potential energy compared with a straight object of the same length. Features that consider the amount of stored potential energy in an actual vessel as compared with a straight vessel of the same characteristics and length may be used. This description refers to these features as “bending energy” features.

There are a number of ways to model or calculate bending energy from the segmented vasculature. In the simplest form of the analysis, bending energy can be calculated as in Equation (1) below:

$${E}_{b} = \frac{1}{2} \int {K ( x )}^{2} dx \qquad ( 1 )$$

wherein the integral of Equation (1) is taken over the length of the vessel and κ(x) is the curvature at each point along the vessel.

As those of skill in the art will note, this approach considers only the curvature of the vessel. Techniques of mechanical engineering and continuum mechanics can be used to model the bending energy more precisely. For example, a more sophisticated model may consider the radius of the vessel and estimate a wall thickness, modeling the vessel as a tube. Strain energy and shear stress could then be calculated, and if calculated, would be considered to be within the category of bending energy features.

As with all of the features described above, statistics like mean, median, standard deviation, range, percentiles, skewness, and kurtosis may be used to describe features like torsion, inflection points, and bending energy in the aggregate, and are typically considered to be features in their own right.

In any particular application that uses vessel morphology features, a single feature or type of feature may be used, or multiple features may be used. As the description above bears out, multiple features may initially be explored for use in any application. Ultimately, various approaches may be used to select the most discriminative (i.e., most relevant), least redundant, and/or most stable features. However, at least initially, multiple, distinct approaches to characterizing vessel morphology may be used in extracting features. For example, vessel tortuosity, torsion, inflection point, and bending energy features may all be extracted from a segmented vasculature.

In general, radiomic and pathomic features cannot be extracted mentally or using pencil-and-paper approaches. Many radiomic and pathomic features are sub-visual and cannot be perceived by the human eye. In the case of vessel morphology, while large-magnitude twists, bends, and changes in direction may, in some cases, be visible to the human eye, at least in a two-dimensional or three-dimensional segmentation, it is often useful or necessary to extract and consider vessel morphology features that are too subtle to be obvious to a human observer. Moreover, even when an individual calculation necessary for feature extraction may be straightforward, as a practical matter, the volume of features extracted across the vasculature to make a viable (i.e., reliable) medical prediction is too vast for manual calculation.

In using these features, as described above, features from the vasculature surrounding a lesion may be compared with the same features extracted from healthy vasculature.

Use in Clinical Trials and with Investigational Treatments and Agents

These tools could also be leveraged many ways in a clinical trial setting. For example, an enrollee in a clinical trial may be required, as part of the enrollment criteria, to have a biomarker that places that enrollee in a particular phenotypical group. As another example, a clinical trial may be structured with a primary, secondary, or exploratory endpoint that involves a particular phenotype or phenotypes. For example, a clinical trial may be structured to test whether the treatment in question is effective in patients having a particular tumor phenotype. Additionally, the types of biomarkers, phenotypes, and assays described here could be used to provide evidence that a particular mechanism underlies a demonstrated clinical effect. For example, while improved overall survival could be a primary endpoint of a clinical trial, a secondary endpoint might involve specific changes in tumor vessel morphology, as measured radiomically using a vessel morphology feature or features.

Biomarkers and phenotypes may also be used more generally to determine which patients are likely to derive greater benefit from an investigational treatment or agent and which are likely to derive a lesser benefit. In some cases, those who will derive greater benefit are responders, and those who will derive lesser benefit are non-responders.

Additionally, once treatment has begun, changes in phenotype can be used to provide early indications that a patient, or a group of patients of the same phenotype, is or is not responding to a particular treatment.

One particular way in which phenotypes according to embodiments of the invention may be helpful in clinical trial and other investigative contexts is in the determination of appropriate dosing for a drug. Typically, drug-dosing decisions are based on survival data, e.g., OS or PFS. That data takes time to acquire. Basing dosing decisions on the kinds of phenotypes, biomarkers, and assays described above may allow critical decisions to be made much faster.

As one example in the drug-dosing context, a phenotype would be established as described above, e.g., in method 10, using a metric such as OS or PFS as a “ground truth” within a patient population with known long term outcomes. A second patient population exposed to different dosages of the drug in question (for instance, within a dose selection clinical trial) would have routine clinical scans, or scans taken specifically for this purpose, assayed, with the necessary radiomic features extracted from their scans (and, if necessary, pathomic features extracted from pathology medical images). A risk score, such as that described above, could be assigned to patients based on the assay. The magnitude of the risk score following treatment, or changes in the risk score over time, may indicate that a particular dose of the drug is sufficient to induce positive change in a lesion or tumor. Similarly, a dose may be deemed ineffective if the risk score remains stable or increases. The most appropriate dose of the drug may be established based on the risk score in combination with other considerations, such as toxicity and side effects. Of course, a risk score is not required in all embodiments.

A risk score derived from a particular, established phenotype could be used “as is” for these purposes, or the phenotype could be optimized for sensitivity to a particular drug or agent, for instance by repeating phenotype discovery/clustering in a population of imaging data that has been exposed to that agent.

In the above description, the phenotypes are established, and scores are used, for vasculature associated with lesions or tumors. In some embodiments, a vessel morphology score may be calculated for any vasculature apparent in a medical image, even if that vasculature is not associated with a lesion or is not apparently associated with a lesion. A phenotype, used in this way to generate a vessel morphology risk score for vasculature that is not apparently associated with a lesion, may be used for a variety of predictions, including diagnostically, to indicate that a disease may be or is likely present, even if a lesion is not apparent; or predictively, to predict the risk or likelihood that a disease will develop.

Systems and methods according to embodiments of the invention may stop at assaying and/or providing a report 300, 350 with a risk score or other indication of the phenotype of a patient's vessel morphology. However, in some embodiments, a method may include the step of administering, by a qualified person, a drug or other therapy, or at least, placing or causing an order to be placed to administer such a drug or other therapy. For example, the method may involve ordering the administration of or administering an immune checkpoint inhibitor, an anti-angiogenic agent, or a chemotherapy agent.

In general, in clinical trials, phenotypes established using radiomic vessel morphology features may also be used to determine the safety, efficacy, and mechanism of action of a drug. With respect to safety, radiomic and pathomic methods may be used to predict certain adverse events, such as pneumonitis, hyperprogression, and liver toxicity, to name a few. In some cases, methods like method 10 may find associations between particular sets of features and particular adverse outcomes when a biomarker is established. In other cases, particular component vessel morphology scores (which will be described below in more detail) may be associated with adverse outcomes. In yet other cases, supervised machine learning may be used, and a machine learning model may be trained to predict certain specific adverse effects based on the extracted radiomic vessel morphology features or some other combination of features.

### Component Vessel Morphology Scores

As was described above, vessel morphology includes a number of different types of vessel morphology characteristics, including, for example, vessel curvature, tortuosity, number of vascular branches, branch length, vascularization, and vascular volume. The above description generally assumes that the measures of vessel morphology, and the features that are extracted from medical images, involve vessel morphology features from more than one category. That is, the vessel morphology scores described above may be calculated using some combination of curvature, tortuosity, vascular branch count, branch length, vascularization, and vascular volume features, or, at least, more than one of those categories.

That need not be the case in all embodiments. In some embodiments, and for some applications, it may be useful or desirable to focus on a “component” vessel morphology score, i.e., a vessel morphology score based on a limited number of types of features. In many cases, a component vessel morphology score may focus on a single category of vessel morphology features. That component vessel morphology score may be used at the outset of treatment and throughout the treatment process, just as described above. In some cases, a separate component score may be calculated for each available type of vessel morphology feature, and all of those scores may be used as biomarkers, with the result potentially allowing a clinician to appreciate the effects of treatment on the various aspects of vessel morphology. In some cases, one vessel component score, e.g., a vessel curvature morphology score, may be more predictive of treatment response or other outcomes than either a composite vessel morphology score or other types of component scores.

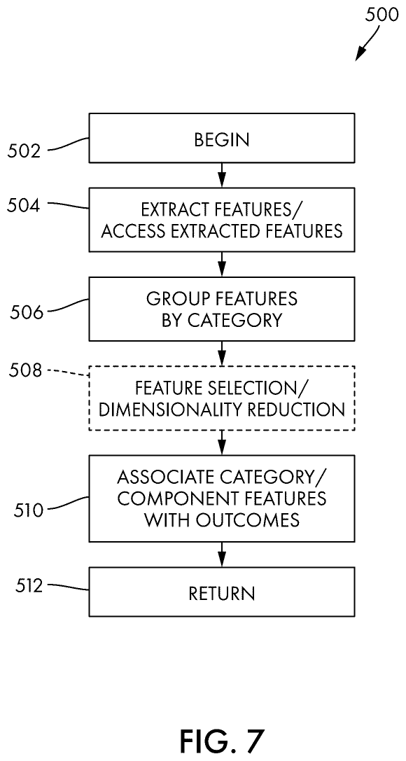

FIG. 7 is a flow diagram of a method, generally indicated at 500, for establishing a phenotype or phenotypes based on a component vessel morphology score. Method 500 is generally similar to method 10 described above, is presented in simplified form for ease of explanation, and begins with task 502. In task 504, method 500 either extracts radiomic vessel morphology features from a medical image or series of medical images, or accesses vessel morphology features that have already been extracted elsewhere.

Following extraction, or accessing already-extracted features, method 500 continues with task 506, and features are grouped according to category or type. For example, features may be grouped into categories such as branching, curvature, torsion, radius, vessel volume, and vessel inflection points. In this embodiment, the grouping is done manually. Other options will be described below in more detail. Method 500 may optionally continue with task 508.

In FIG. 7, task 508 is outlined by broken lines, indicating that it is an optional task. In task 508, dimensionality reduction and feature selection techniques may be used to reduce the number of features used in each category. This may simplify the application of the component biomarker once established. Techniques such as principal component analysis and the mRMR approach may be used in task 508. However, dimensionality reduction and/or feature selection may be not necessary in all embodiments or for all applications. Rather, a panel of features may simply be used without regard to their number or relevance.

After task 508 is complete, or after it is decided not to perform dimensionality reduction and/or feature selection after task 506, a panel of features is available for use for at least one type of vessel morphology feature. Typically, the tasks of method 500 are repeated for each type of vessel morphology feature that is being evaluated or used, until a set of features is defined for each component or type of vessel morphology features. Method 500 continues with a process of associating those component sets of vessel morphology features with outcomes in task 510.

Once the component feature sets are associated with outcomes in task 510, method 512 terminates at 512. When method 500 terminates, each component feature set is, in essence, a biomarker associated with at least one outcome. If a component feature set is not clearly associated with an outcome in task 510, it may not be used as a biomarker at all, it may be used in conjunction with other component feature sets that are more clearly associated with outcomes in order to provide greater context or more information on the evolution of a tumor through the treatment process, or it may be used regardless of its lack of association with a particular outcome to monitor change longitudinally through a course of treatment, with the potential for understanding more about the treatment's safety, efficacy, or mechanism of action.

Variations on the scheme of method 500 are possible and potentially numerous. For example, in method 500, features are first divided manually into categories. Instead of doing that, clustering, dimensionality reduction, and other techniques could be first used on a composite vessel morphology score to establish components and those results used to define sets of features for component scores.

Component vessel morphology scores may be used for other purposes as well, both in clinical practice, in research environments, and in clinical trial work. For example, in clinical trials, component vessel morphology scores may be used once treatment is underway to assess, after administering the investigative and/or placebo treatment(s) or longitudinally during treatment, which aspects of the vessel morphology changed in response to those treatments. This, in turn, may help clinicians, researchers, and others to understand not only the overall efficacy of the treatment(s), but also the mechanism of action of those treatments. For example, if vessel morphology studies or scores indicate that there are fewer vessels, fewer vascular branches, or smaller vessels, on average, the treatment(s) may be understood, at least in some contexts, to have anti-angiogenic effects. By contrast, if component vessel morphology scores show the vasculature to be less tortuous, to have fewer inflection points, or to have less bending energy, the mechanism of action may be understood as reducing vascular disorganization. While assessments of the safety, efficacy, or mechanism of action of a drug may be made in single patients or in small groups of patients, it may be most useful in these sorts of inquiries to consider the values of the vessel morphology features or vessel morphology scores from larger populations of patients.

### Interfaces and Data Integration

As may be apparent from the above, the output(s) from the kinds of methods and systems described here may run the gamut from simple to complex. In its simplest form, for example, a composite vessel morphology score or component score may be reported in numerical form with little to no context. Composite and component scores may also be reported with the kind of context shown in the reports 300, 350 of FIGS. 5-6. However, just as the outputs of these methods may be simple, they may also be more complex and integrated with other data to give clinicians, researchers, and others more insight into how a patient's disease is evolving and responding or not responding to treatment.

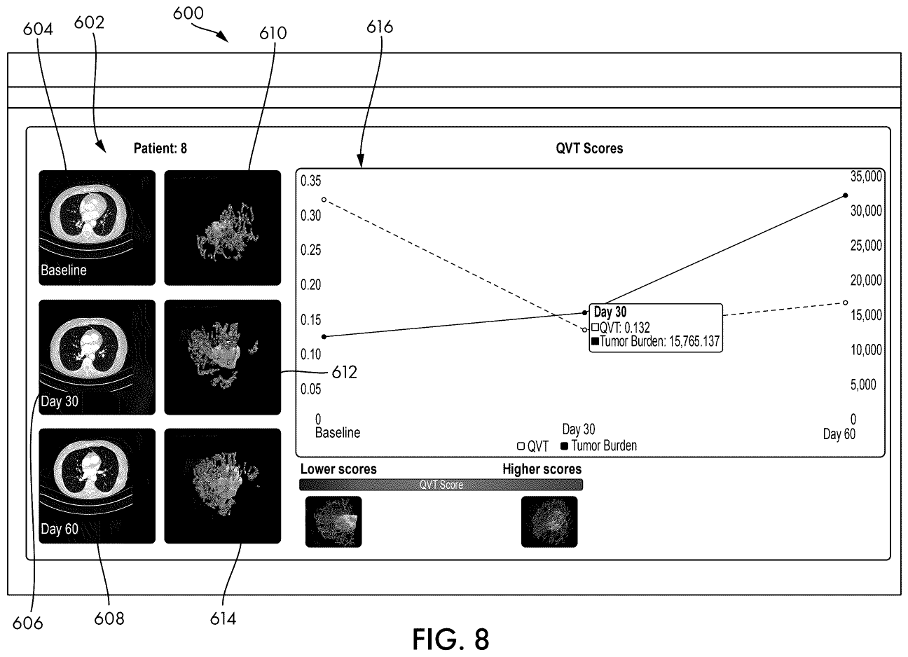

FIG. 8 is an illustration of a graphical user interface (GUI), generally indicated at 600, that combines vessel morphology component and composite scores with other data to provide a more complete understanding of a patient's condition. In the illustration of FIG. 8, the GUI 600 explores the progress of one patient's disease at time zero (i.e., at the time of diagnosis), at 30 days after diagnosis, and at 60 days after diagnosis. In this example, the patient is suffering from NSCLC, and although the patient's diagnosis may influence exactly what kind of information is displayed and how it is displayed, ultimately, an appropriately-informative GUI similar to the GUI of FIG. 8 can be constructed for essentially any diagnosis.

In the GUI 600, an imaging panel 602 to the left of the GUI 600 displays at least one relevant slice 604 of the patient's chest CT on day 0, at least one relevant slice 606 of the patient's chest CT on day 30, and at least one relevant slice 608 of the patient's chest CT on day 60. Horizontally adjacent to each chest CT slice 604, 606, 608 in the imaging panel is an image 610, 612, 614 illustrating the machine-established segmentation of the tumor and its vasculature at the same point in time. The images 610, 612, 614 illustrating the segmentation of the tumor and its vasculature may be annotated with a color or grayscale gradient indicating, for example, how a component vessel morphology score or composite vessel morphology score varies along the vasculature.

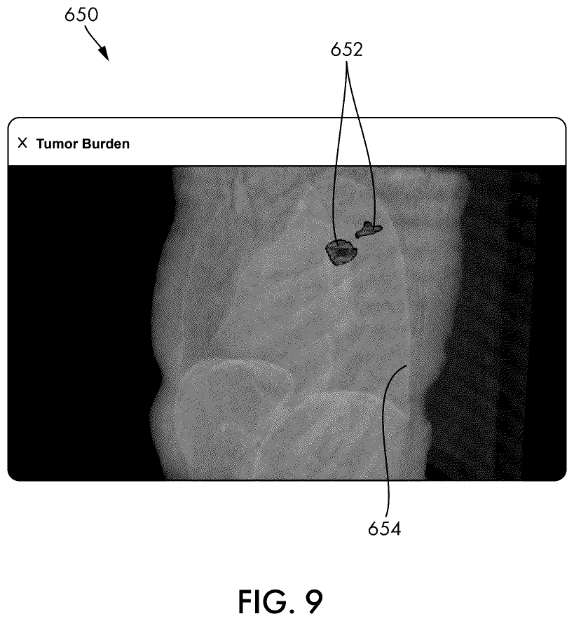

One advantage of the GUI 600 is that clicking on the images 604, 606, 608, 610, 612, 614 may provide the user with additional information. For example, clicking on one of the chest CT image slices 604, 606, 608 may trigger a three-dimensional illustration or animation showing the position of the tumor from one or several perspectives, using a segmentation established while executing the methods described here. FIG. 9 is an illustration of a pop-up window 650 that illustrates the position and extent of a tumor 652 within an illustration of the chest 654. The illustration of the chest 654 may be constructed from the patient's CT or MRI scans or an X-ray, or it may be a generic illustration of a human chest that is used to express position and extent.

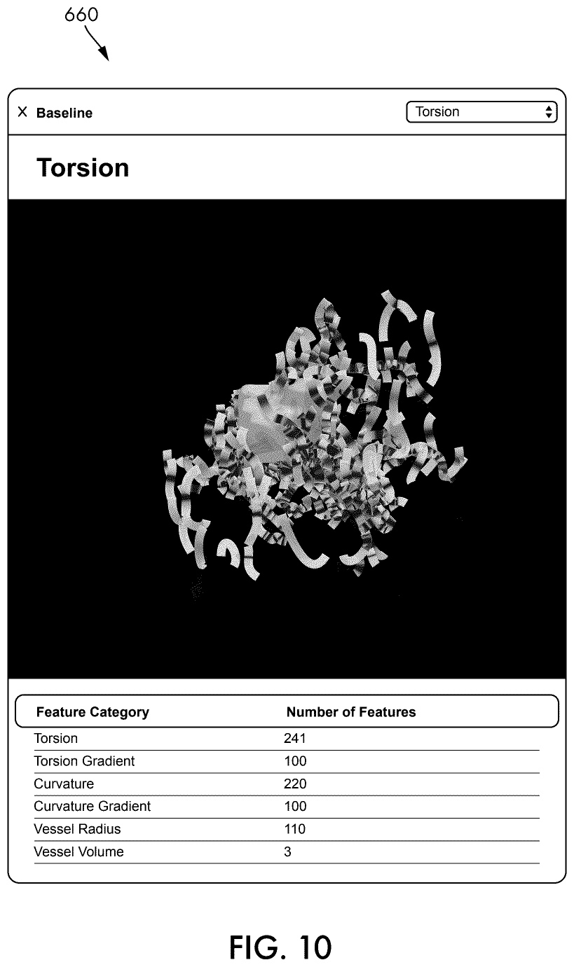

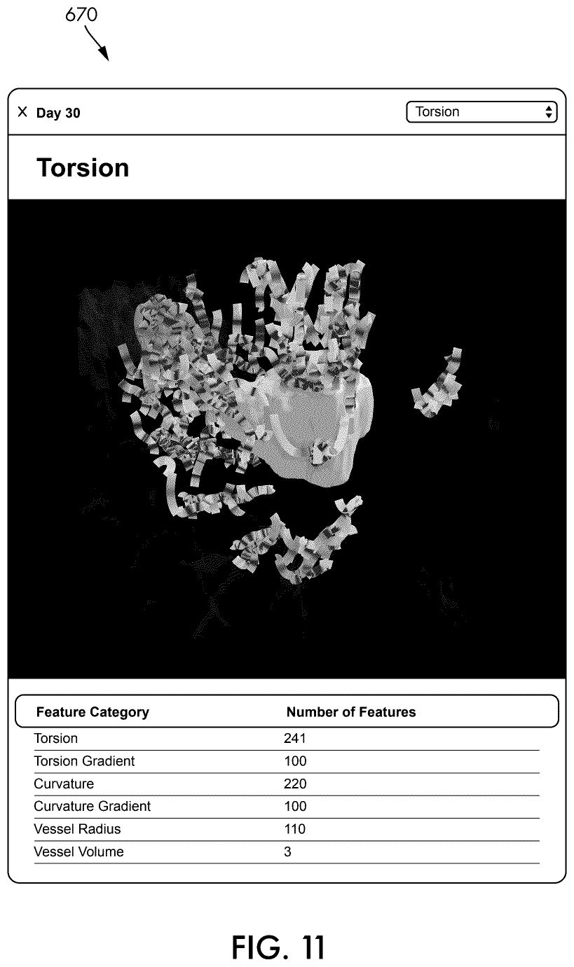

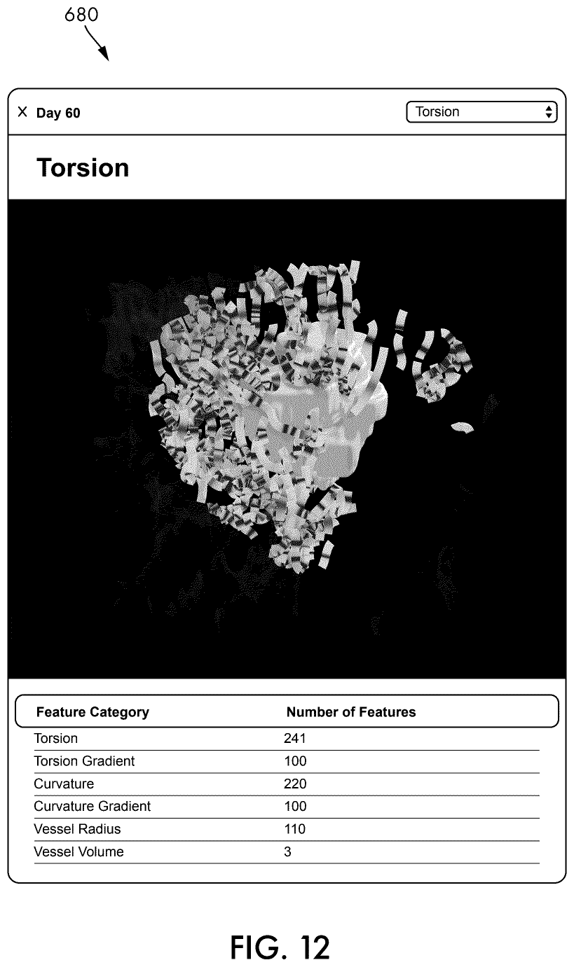

If the user clicks on one of the images 610, 612, 614 illustrating the segmented tumor, the GUI 600 may present an enlarged view of the tumor with additional information. FIGS. 10-12 are illustrations of windows 660, 670, 680 that illustrate the tumor 652 at the various points in time with its vasculature annotated using a gradient to show the value of the component or composite vessel morphology score.

In addition to information about the tumor, in this implementation, the GUI 600 also displays a composite vessel morphology score (marked in the illustration of FIG. 8 as a “QVT score”) against another known metric: tumor burden. This is done in a central graph 616 and provides additional context and an association with a metric that is known and familiar to clinicians.

Of course, as those of skill in the art will understand, the ways in which data from the methods and systems described here can be visualized are essentially unlimited. FIG. 8 and the other figures indicated here provide only one example of a GUI 600 that integrates the outputs of the methods and systems with source material (i.e., CT scans) and intermediate products of the methods (i.e., three-dimensional tumor and vasculature segmentations) with other metrics (in this case, tumor burden).

### Interventions and Procedures

In much of this description, the term “medical treatment” refers to treatment with a drug, such as a chemotherapy or immunotherapy treatment, an anti-angiogenic, etc. However, the range of medical treatments that can be supported or informed by vessel morphology studies and scores is not so limited. In at least some embodiments, vessel morphology studies, vessel morphology scores, component or “panel” vessel morphology studies and scores, and other such information derived from radiomic vessel morphology features may be used to support, predict, and inform a wide variety of physical, surgical, and radiotherapeutic interventions.

In general, vessel morphology studies, scores, and other elements according to embodiments of the present invention may be used whenever vessel morphology is a factor in planning, executing, or monitoring a procedure or procedures. For example, in an embolization procedure, such as a transarterial radioembolization (TARE), vessel morphology studies, scores, or component scores could help to identify patients who are good candidates for the procedure by identifying patients with tumors with relatively organized, uncomplicated lesion-associated vasculature. Vessel radii and other component vessel morphology features may facilitate understanding of which vessels are suitable for embolization and which are not, and whether there are particular vessels that cannot be embolized or otherwise treated with the techniques being considered.

When a proposed treatment requires vascular access to a tumor, vessel morphology studies and scores may help to evaluate viable vascular access routes through the tumor's vasculature, or whether no viable path exists. The output from such studies may simply indicate a threshold “yes-accessible” or “no-inaccessible” based on a threshold (e.g., a threshold based on the number of vascular branches, the average vessel radius, the average vessel tortuosity, the average vessel bending energy, etc.). Alternatively, the output from such studies may involve using other machine learning models or mathematical algorithms to display an appropriate vascular path for a particular intervention, given a three-dimensional segmentation of the vasculature. In these cases, vessel morphology results or scores may be reported not only for the whole tumor, but for portions of it, e.g., divided into quadrants or relative to some defined coordinate system, either with an origin within the tumor or with an origin relative to some other bodily landmark.

In radiotherapy, vessel morphology studies and scores may be used to plan externally-applied radiation doses or to plan the placement of internal radioactive seeds in brachytherapy. For example, areas of a tumor showing greater vascular disorganization, or, in a case of multiple lesions or tumors, lesions or tumors showing greater vascular disorganization, may be more heavily targeted for radiotherapy. Alternatively, if vessel morphology scores indicate that a particular tumor has only a few large vessels that are relatively non-tortuous and are supplying the tumor or lesion, those vessels may be targeted for radiotherapy, embolization, or other such interventions.

Vessel morphology studies and scores may also be used to plan surgical procedures like tumor resection or partial resection, provide insight on tumors or portions of tumors that may be the highest priority for resection, and provide insight on any special concerns or difficulties that may be encountered in ablating, cauterizing, clamping, or otherwise managing the vasculature during a surgical procedure.

Surgical treatments and interventions that may be aided by vessel morphology studies and scores may also involve precise delivery of drugs to particular lesions or portions of a lesion, either in fluid form or in the form of drug-eluting polymers and polymeric gels, pellets, microspheres, microbeads, microarrays, implantable polymeric matrices, etc. These latter types of drug-delivery systems tend to remain more static when placed; thus, planning their placement can be helpful.

As those of skill in the art will realize, the kinds of visualizations described above and shown, e.g., in FIGS. 8 and 10-13B, may be useful in communicating the results of vessel morphology studies and scores for interventional visualization. Color gradients and other such visual indicators of vessel morphology scores and feature values may be used, e.g., to show vessels of particular concern for a specific type of procedure, to show average vessel properties, e.g., average radius, average tortuosity, etc. If a generative machine learning model is used to create further annotations of, e.g., a segmentation of a lesion, those may be overlaid on the kind of interface shown in the figures.

While this description focuses specifically on vessel morphology and its applications, other techniques may be used in conjunction with vessel morphology, particularly in the areas of intervention planning and execution. For example, a number of techniques may be used, in combination with radiomic vessel morphology, to further understanding of blood flow within the vasculature, including Doppler ultrasound, dynamic contrast enhanced MRI (DCE-MRI), positron emission tomography (PET), and traditional angiography techniques.

### EXAMPLES

### Example 1

577 vessel morphology features describing vessel curvature, tortuosity, number of vascular branches, branch length, vascularization, and vascular volume were extracted from CT scans prior to ICI monotherapy of a cohort of 406 patients with advanced NSCLC. Phenotypes were discovered using unsupervised dimensionality reduction (t-SNE) and clustering (k-means).

Phenotypes were tested for outcome associations and independence from clinical variables in a subset of 186 patients. We identified two discrete outcome-associated phenotypes of tumor vascularity: QVT-High was characterized by chaotic vascular shape: increased tumor vessel torsion, curvature, volume, and radius. QVT-Low had fewer variations in vessel shape and size across the tumor vasculature. QVT-High was significantly associated with adverse outcomes: OS (HR=1.6 (1.1-2.3), p=0.02) and PFS (HR=1.8 (1.3-2.5), p<0.005), independent of patient age, gender, histology, and PD-L1 expression (negative, low, high) in a multivariable evaluation.

These findings suggest that tumor vascularity plays a critical role in NSCLC outcomes. We introduce a unique and non-invasive biomarker approach that could assist in identifying patients eligible for ICI/VEGF dual agents and improve monitoring.

### Example 2

In a discovery cohort of 375 NSCLC patients, an unsupervised clustering model of interpretable vessel morphology features—including vessel curvature, twistedness, and branching—was utilized to create an automated and continuous 0-to-1 vessel morphology score indicating the degree of high risk elevated vascularity. The vessel morphology score was computed and validated for association to OS using pre-treatment (n=266) and first on-treatment (n=143) CT from 266 ICI monotherapy recipients. For patients with pre-treatment histopathology samples available (n=31), deep learning models were used to compute the proportions of various cellular subpopulations, which were then correlated with vessel morphology score.

Higher vessel morphology score was associated with shorter OS at the baseline (HR: 2.07, p=0.019) and first on-treatment (HR: 4.22, p=0.00083) scans, as was longitudinal change (HR=2.89, p=0.0056). Stratifying patients by the directionality of change also stratified OS (HR=1.91, p=0.0017) with median OS of 10 months for Increasing and 22 months for Decreasing. These groups remained independently prognostic when adjusted for RECIST best overall response (p=0.0340) and volume change (p=0.0015). Higher baseline QVT Score was linked to a hypoxic microenvironment, as indicated by a significant association with necrotic cell proportion (r=0.40, p=0.024) on histopathology (Table 1).

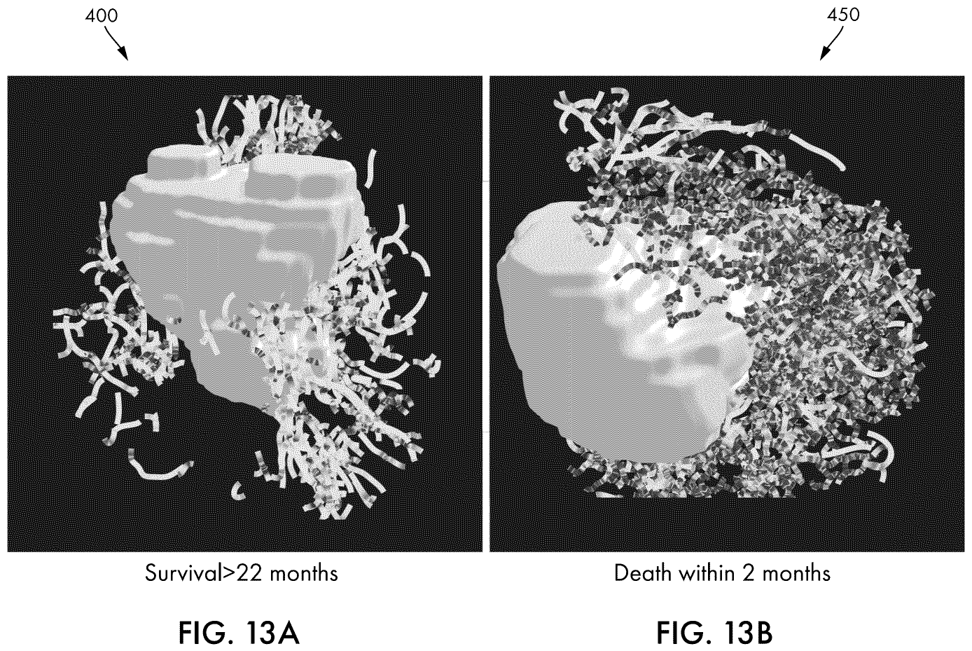

FIG. 13A is an illustration of a vessel morphology 400 associated with NSCLC having a lower vessel morphology score, with which the patient had an OS greater than 22 months. FIG. 13B is an illustration of a vessel morphology 450 associated with NSCLC having a higher vessel morphology score, with which the patient died within 6 months. In this particular example, some of the additional complexity of the vessel morphology 450 with the higher vessel morphology score can be appreciated qualitatively with the naked eye. As those of ordinary skill in the art will understand, the clear visual difference between the two vessel morphologies 400, 450 is unusual. The methods and systems described here may distinguish between low-risk and high-risk vessel morphologies based on sub-visual features, or features that otherwise cannot be appreciated qualitatively.

TABLE 1
Correlation of Baseline Vessel Morphology Score
with Cellular Composition on Histopathology.

| | Pearson | |
|---|---|---|
| | Correlation | p-value |
| Necrosis | 0.40 | 0.024 |
| Fibroblasts | 0.21 | 0.25 |
| Tumor-infiltrating lymphocytes | 0.23 | 0.21 |
| Tumor nuclei | −0.19 | 0.31 |

### Example 3

Vessel morphology component scores were derived by grouping 910 vessel morphology features comprising a composite vessel morphology score into six biological categories, summarized by principal component analysis. The panel was evaluated in 557 patients from the phase 3 SWOG S0819 trial and a real-world ICI monotherapy cohort (n=147). Vessel morphology composite score/component score changes from baseline to first on-treatment CTs were assessed by paired t-test and substratified by objective response (OR).

While the mono-chemotherapy showed no vascularity changes, VEGFi regimens reduced vessel morphology score, with Branch and Curvature Component Scores decreasing significantly but Vessel Volume unchanged. In contrast, ICI recipients exhibited significant increases in composite vessel morphology score and 5 of 6 component scores, showing opposite changes to VEGFi in four component scores and differing effects on vessel Radius and Volume. VEGFi produced the greatest vascularity decrease in patients achieving OR, with milder decreases in non-OR. For ICI, OR showed negligible vessel morphology change, while vascularity significantly increased in non-OR across all component scores except Radius. Table 2 below summarizes the results.

TABLE 2
Results of Example 3

| | S0819 arm1: | S0819 arm2: | S0819 arm3: | S0819 | |
|---|---|---|---|---|---|
| | mono | chemo + | chemo + | arm4: | |
| Treatment | chemotherapy | cetuximab | bevacizumab | chemo + | mono |
| Group | (chemo) | (cet) | (bev) | cet + bev | ICI |
| N | 170 | 157 | 116 | 114 | 147 |
| Composite | — | — | Down | Down | Up |
| Score | | | | | |
| Component 1: | — | — | Down | Down | Up |
| Branching | | | | | |
| Component 2: | — | — | Down | Down | Up |
| Curvature | | | | | |
| Component 3: | — | — | Down | No | Up |
| Torsion | | | | Trend | |
| | | | | (down) | |
| Component 4: | — | — | Down | — | — |
| Radius | | | | | |
| Component 5: | — | — | — | — | Up |
| Vessel Volume | | | | | |
| Component 6: | — | — | Down | No | Up |
| Inflection | | | | Trend | |
| Points | | | | (down) | |

Note:
Down/Up p <= 0.05; No Trend (down) 0.05 < p < 0.1.
— a dash (—) indicates no trend

All patents and non-patent references cited herein are hereby incorporated by reference in their entireties.

While the invention has been described with respect to certain embodiments, the description is intended to be exemplary, rather than limiting. Modifications and changes may be made within the scope of the invention, which is defined by the appended claims.

What is claimed is:

1. A method, comprising:
using machine learning, grouping first medical images from a group of patients into two or more groups on the basis of a set of radiomic vessel morphology features extracted from the first medical images or from a vascular segmentation derived from the first medical images; and
on the basis of said grouping, defining a phenotypical set of radiomic vessel morphology features, the phenotypical set of radiomic vessel morphology features being (1) at least a subset of the set of radiomic vessel morphology features extracted from the first medical images, and (2) medically predictive of the two or more groups.

2. The method of claim 1, further comprising:
extracting the phenotypical set of radiomic vessel morphology features from a second medical image or images of a patient, or from a vascular segmentation derived from the second medical image or images;
based on values of the extracted phenotypical set of radiomic vessel morphology features, making a medical prediction concerning the patient.

3. The method of claim 2, wherein the first set of medical images indicate at least one lesion, and the set of radiomic vessel morphology features are extracted from vasculature associated with the at least one lesion.

4. The method of claim 3, wherein the second medical image or images of the patient indicate at least one patient lesion, and the phenotypical set of radiomic vessel morphology features are extracted from vasculature associated with the at least one patient lesion.

5. The method of claim 2, further comprising one or more of:
choosing or determining a course of treatment for the patient based on the medical prediction;
based on the values of the extracted phenotypical set of radiomic vessel morphology features, determining the efficacy of the course of treatment or another course of treatment; or
based on the values of the extracted phenotypical set of radiomic vessel morphology features extracted after a medical treatment has been administered to the patient, determining a mechanism of action of the medical treatment.

6. The method of claim 2, wherein said making the medical prediction concerning the patient comprises:
deriving a vessel morphology score based on the values of the extracted phenotypical set of radiomic vessel morphology features; and
outputting the score.

7. The method of claim 6, wherein said outputting comprises generating a report in which the score is given relative to scores associated with the known outcomes.

8. The method of claim 1, wherein said using machine learning comprises using unsupervised machine learning.

9. The method of claim 8, wherein the clustering comprises consensus clustering.

10. The method of claim 2, wherein the second medical image or images comprise computed tomography (CT) or magnetic resonance imaging (MRI) scans.

11. The method of claim 1, wherein the phenotypical set of radiomic vessel morphology features comprises one or more categories of radiomic features selected from the group consisting of branching features, torsion features, curvature features, radius features, vessel volume features, inflection point features, features derived from Frenet-Serret frame vectors, and bending energy features.

12. The method of claim 11, wherein the phenotypical set of radiomic vessel morphology features comprises two or more categories of the radiomic features.

13. The method of claim 11, wherein the phenotypical set of radiomic vessel morphology features comprises one category of the radiomic features.

14. A method, comprising:
extracting a set of phenotypical radiomic vessel morphology features from (1) a three-dimensional segmentation of vasculature, (2) a portion of the three-dimensional segmentation of the vasculature, or (3) a transform or projection of the three-dimensional segmentation of the vasculature, the three-dimensional segmentation of the vasculature being constructed from one or more medical images of a patient;
based on values of features within the extracted phenotypical set of radiomic vessel morphology features, evaluating safety, efficacy, or mechanism of action of a medical treatment in the patient.

15. The method of claim 14, wherein the patient has a lesion shown in the one or more medical images, and vasculature segmented to create the three-dimensional segmentation of vasculature is associated with the lesion.

16. The method of claim 15, further comprising performing said extracting on medical images from a population of patients; and
performing said evaluating using the sets of phenotypical radiomic vessel morphology features from the medical images from the population of patients.

17. The method of claim 15, further comprising:
performing said extracting on medical images from a first population of patients and from medical images from a second population of patients, the first population of patients and the second population of patients having undergone different medical treatments; and
evaluating the safety, the efficacy, or the mechanism of action of the different medical treatments based on values of radiomic vessel morphology features from said extracting.

18. The method of claim 15, further comprising, based on the values of the features within the extracted phenotypical set of radiomic vessel morphology features, forming a vessel morphology score and performing said evaluating, at least in part, on the basis of the vessel morphology score.

19. The method of claim 18, further comprising providing the vessel morphology score in a report along with score-based guidelines.

20. A method, comprising:
extracting one or more component sets of phenotypical radiomic vessel morphology features from (1) a three-dimensional segmentation of vasculature, (2) a portion of the three-dimensional segmentation of the vasculature, or (3) a transform or projection of the three-dimensional segmentation of the vasculature, the three-dimensional segmentation of the vasculature being constructed from one or more medical images of a patient, each of the one or more component sets of phenotypical radiomic vessel morphology features including only a specific category or type of radiomic vessel morphology features;
creating a vessel morphology score for each of the one or more component sets of phenotypical radiomic vessel morphology features based on the values of the vessel morphology features therein;
using the vessel morphology scores for at least some of the one or more component sets of phenotypical radiomic vessel morphology features to evaluate the safety, efficacy, or mechanism of action of a treatment for the patient.

21. The method of claim 20, wherein the vasculature is associated with a lesion or tumor.

22. The method of claim 20, further comprising, based on the efficacy, selecting a course of treatment for the patient.

23. The method of claim 22, further comprising:
after administering the course of treatment to the patient, repeating said extracting and said creating using a three-dimensional vascular segmentation established based on one or more medical images taken after said administering to establish a second set of the vessel morphology scores; and
using the second set of the vessel morphology scores to evaluate safety, efficacy, or mechanism of action of the course of treatment.

24. The method of claim 20, wherein the specific categories or types of radiomic vessel morphology features are selected individually from the group consisting of branching features, torsion features, curvature features, radius features, vessel volume features, inflection point features, features derived from Frenet-Serret frame vectors, and bending energy features.
